(19) United States
(12) Patent Application Publication
(10) Pub. No.: US 2013/0190864 A1
(43) Pub. Date: Jul. 25, 2013

(54) ANNULOPLASTY PROSTHESES AND SURGICAL TECHNIQUES

(71) Applicant: Aram K. Smolinsky, Netanya (IL)

(72) Inventor: Aram K. Smolinsky, Netanya (IL)

(21) Appl. No.: 13/813,029

(22) PCT Filed: Aug. 11, 2011
(86) PCT No.: PCT/IL11/00656

§ 371 Date: Apr. 9, 2013

Related U.S. Application Data
(60) Provisional application No. 61/372,618, filed on Aug. 11, 2010.

(51) Int. Cl. A61F 2/24

(52) U.S. Cl. CPC A61F 2/2442
USPC 623/2.36

(57) ABSTRACT

An annuloplasty prosthesis is provided that includes a skeleton and a sleeve. The skeleton comprises a rigid posterior bar segment, which has first and second bar ends. The sleeve (a) comprises a flexible and substantially non-extensible fabric material, (b) covers the rigid posterior bar segment, (c) includes a first portion that extends at least 20 mm beyond the first bar end and a second portion that extends at least 25 mm beyond the second bar end, and (d) has a plurality of visually-sensible fiducial designators at respective longitudinal locations distributed along at least a portion of the sleeve, which fiducial designators are arranged to indicate a longitudinal center of the rigid posterior bar segment. The prosthesis is rigid along the rigid posterior bar segment, and is flexible or semi-rigid along at least 75% of the first portion of the sleeve and along at least 75% of the second portion of the sleeve.

### CROSS-REFERENCE TO RELATED APPLICATIONS

The present application claims priority from U.S. Provisional Application 61/372,618, filed Aug. 11, 2010, which is assigned to the assignee of the present application and incorporated herein by reference.

### FIELD OF THE APPLICATION

The present application relates generally to implantable medical devices and surgical techniques, and specifically to annuloplasty bands or rings for surgical correction of atrioventricular valves of the human heart, and surgical techniques for treating the mitral valve.

### BACKGROUND OF THE APPLICATION

In mitral valve annuloplasty, a prosthetic ring or band is implanted to support and reduce the diameter of the annulus of an atrioventricular valve (the mitral valve or tricuspid valve) that suffers from valvular insufficiency or other dysfunction. Mitral valve annuloplasty (with or without repair of the leaflets and chordae tendineae) has become an accepted and desirable alternative to valve replacement. The annuloplasty ring reduces the pathologically dilated circumference of the native annulus, in order to restore proper valve leaflet coaptation.

Alain Carpentier was a leader in the development of the rigid annuloplasty ring. See, for example, Carpentier A et al., “A new reconstructive operation for correction of mitral and tricuspid insufficiency,” J Thorac Cardiovasc Surg 61(1):1-13 (1971). Carlos Duran was a leader in the development of the totally flexible ring. See, for example, Duran CG et al., “Clinical and hemodynamic performance of a totally flexible prosthetic ring for atrioventricular valve reconstruction,” Ann Thorac Surg 22:458-463 (1976). A comparison of these two types of bands is presented by Okada Y et al., in “Comparison of the Carpentier and Duran prosthetic rings used in mitral reconstruction,” Ann Thorac Surg 59(3):658-62 (1995).

More recently, posterior annuloplasty bands have been developed, including the Cosgrove-Edwards® annuloplasty band, which includes a flexible piece of material for use only in the posterior portion of the annulus from trigone to trigone.

The CG Future® Band (Medtronic, Inc., Minneapolis, Minn.) is described as remodeling the mitral annulus while supporting physiologic annular movement. The CG FUTURE® Composite Annuloplasty Ring (Medtronic, Inc.) is described as providing semi-rigid posterior remodeling with anterior flexibility.

The first-generation ring prostheses were described as having a flat two-dimensional configuration. Later, Main Carpentier recommended bending the anterior part of the rigid ring upward (to pull it away from the subaortic space) in the “Le Club Mitral” sessions, and several other three-dimensional rings, mostly for ischemic mitral insufficiency, were presented later. The saddle shape of the native mitral valve was found to reduce systolic strain on the middle scallop (P2) of the posterior mitral leaflet (see Padala M et al., “Saddle Shape of the Mitral Annulus Reduces Systolic Strains on the P2 Segment of the Posterior Mitral Leaflet,” Ann Thorac Surg 2009; 88:1499-505). Medtronic, Inc., inspired by this concept, introduced the Profile 3D™ annuloplasty system for mitral valve repair.

Systolic anterior motion (SAM) occurs after some mitral valve repair operations. SAM causes subaortic stenosis (SAS), and may induce mitral regurgitation. The conventional techniques to prevent SAM demand reduction in the height of the posterior mitral leaflet. Some techniques developed to reduce SAM involve cutting, resecting, and sewing the leaflet to achieve the reduction. Non-cutting techniques have been described, like the folding plasty, and a combination of cutting and folding.

### SUMMARY OF APPLICATIONS

Some applications of the present invention provide techniques for sizing an annuloplasty prosthesis for an individual patient's mitral anatomy. A surgeon measures a height of an anterior leaflet of a native mitral valve. Responsively to the measured height, an appropriately-sized annuloplasty prosthesis is selected from a plurality of differently-sized annuloplasty prostheses, and implanted at the native mitral valve. For some applications, as described in more detail below, the surgeon additionally measures a height of the posterior leaflet, and the annuloplasty prosthesis is sized based on the heights of both the anterior and posterior leaflets, in order to provide a full-sized prosthesis. Typically, an inter-trigone distance between lateral and medial fibrous trigones of the native mitral valve is also measured, and the appropriately-sized annuloplasty prosthesis is selected responsively to both the measured height(s) and the measured inter-trigone distance.

These independent measurements enable the surgeon to select a valve prosthesis having an anterior-posterior diameter most appropriate for the patient's native mitral valve, independent of the inter-trigone distance. Such precise two-dimensional sizing of the valve prosthesis avoids a mismatch between the anterior leaflet height and the height of the prosthesis, and may reduce the risk of systolic anterior motion (SAM). For some applications, the plurality of differently-sized annuloplasty prostheses include at least two prostheses that have a same side-to-side width, and different respective anterior-posterior heights.

It is noted that the anatomical features of native valves generally have different proportions from one another, and not only different sizes. The inventor believes that conventional annuloplasty prostheses are provided in sizes that do not reflect these different proportions, based on the incorrect assumption that the optimal functional anatomical proportions of native mitral valves are uniform. In contrast, kits provided by some applications of the present invention typically include prostheses having dimensions that separately accommodate the inter-trigone distance and the height of the anterior leaflet, and, optionally, the height of posterior leaflet. It is emphasized that some of the kits described herein include the novel annuloplasty prostheses described herein, while others of the kits described herein include conventional annuloplasty prostheses.

Conventional open-heart annuloplasty procedures are typically single-leaflet repair procedures, which restore proper function only to the anterior leaflet. The annuloplasty prosthesis is sized to support typical anterior leaflets, and a segment of the posterior leaflet may be resected to remove any portion of the posterior leaflet considered redundant. After the procedure, the posterior leaflet serves mainly to provide a coaptation countersurface for the functional anterior leaflet. If the height of the posterior leaflet is measured at all, the measurement is made in order to ascertain how much the posterior leaflet needs to be reduced.

While this conventional procedure often produces acceptable results, it has at least two disadvantages. First, this conventional procedure sometimes causes SAM, as discussed hereinabove in the Background of the Application. Second, this conventional procedure generally produces relatively small valve orifice areas, particularly in patients with posterior leaflet dominance. Post-procedure orifice areas of between two and three cm² are common, compared to orifice areas of about 6 cm² in a healthy valve.

In contrast, techniques of the present invention in which the height of the posterior leaflet is additionally measured restore function to both the anterior and posterior leaflets, in order to provide a full-sized prosthesis. The annuloplasty prosthesis is sized based on the heights of both the anterior and posterior leaflets, and the height of the posterior leaflet is not reduced. This sizing prevents undersized prostheses, measured only according to the anterior leaflet, even when it is relatively small. As a result, the technique does not produce the relatively small orifice areas common in conventional procedure. In addition, the technique provides space in the ventricle for the native valve to expand, thereby reducing the risk of SAM. This technique is particularly appropriate for patients with posterior leaflet dominance.

In order to measure the height of the anterior leaflet, the surgeon pulls the posterior portion of the anterior leaflet in a posterior direction, such as by using a hook or other surgical tool. This stretching and straightening is generally necessary to obtain an accurate measurement, because a portion of the anterior leaflet is drawn down into the ventricle by the chordae tendineae, in order to provide a coaptation surface with a similar coaptation surface near the tip of the posterior leaflet. The coaptation surfaces typically share a common coaptation length. Similarly, in order to measure the height of the posterior leaflet, the surgeon pulls the anterior portion of the posterior leaflet in an anterior direction, such as by using a hook or other surgical tool. This stretching and straightening is necessary to obtain an accurate measurement, for the same reason given above for the anterior leaflet.

An appropriately-sized full-sized annuloplasty prosthesis is selected based on a comparison of the anterior-poster height of the prosthesis with the sum of the anterior leaflet height and the posterior leaflet height, minus twice the coaptation length. In the case of the prosthesis described below having side bars and a posterior bar, the anterior-poster height of the prosthesis is the length of the side bars, typically plus the height of the posterior bar curvature.

The anterior-posterior height of the prosthesis is selected based on the combined height of the portions of the anterior and posterior leaflets that face the atrium. However, as described above, the lengths of these portions are not in practice directly measured. Instead, the heights of anterior and posterior leaflets are measured when the leaflets are stretched. For some applications, in order for the prosthesis to enable the natural leaflets to provide the coaptation surfaces, twice the coaptation length is subtracted from the sum of the measured heights. Because the coaptation length does not vary substantially between patients, and is typically between 8 and 12 mm, a constant can be used to estimate twice the coaptation length. The constant typically has a value between 1.5 and 2.5 cm, such as 2 cm. As intra-operative views and measurements are done during cardioplegia, in a flaccid heart, they may distort the in-vivo anatomy, when the coaptation point of the leaflets is deeper than the annular plane. Thus, for some applications, an additional constant is subtracted to account for the depth of coaptation in the beating heart, such as between 1.5 and 2 mm. A combined constant is typically between 2.0 and 3.0 cm, such as 2.5 cm.

This technique thus provides a full-sized prosthesis, which preserves the full orifice area of the native mitral valve, while maintaining the proper coaptation length and depth, in order to provide full closure of the valve.

In some applications of the present invention, an annuloplasty prosthesis is configured to provide absolute rigidity in the anterior-posterior direction, and high flexibility in the side-to-side direction. The prosthesis includes a skeleton that comprises a rigid posterior bar and two non-extensible side bars. The posterior ends of the side bars are coupled to respective ends of the posterior bar, such that the side bars articulate with the posterior bar at respective articulating joints. For some applications, the coupled ends of the bars are shaped to define rings, which are linked to one another like the links of a conventional metal chain. The anterior ends of the side bars are typically not coupled to any elements of the rigid skeleton.

The rigidity provided in the anterior-posterior direction prevents the slow enlargement of the anterior-posterior diameter of the native valve allowed by some semi-flexible annuloplasty rings. The rigidity of the posterior bar prevents bellowing backwards of the base of the posterior leaflet, and supports the weakest point of the native valve that lacks fibrous support (the native mitral valve does not have a complete fibrous annulus). Such enlargement has been observed to sometimes result in central leaks over time. In addition, the posterior bar has a relatively low burden, and has some freedom of motion, which provides the posterior bar with some shock absorption capability, thereby protecting the posterior bar from breaks.

The prosthesis is typically implanted at a native mitral valve, in order to reduce the pathologically dilated native annulus, and thereby restore proper valve function. The surgeon places the prosthesis at the mitral valve such that the posterior bar is positioned along the middle scallop (P2) of the posterior leaflet, one of the side bars is positioned along the lateral scallop (P1) of the posterior leaflet and extending to the lateral fibrous trigone, and the other side bar is positioned along the medial scallop (P3) of the posterior leaflet and extending to the medial fibrous trigone.

The surgeon couples the articulating joints of the prosthesis to the native junctions (small commissures) of P2: one of the articulating joints is coupled to the mitral valve at the junction of P1 and P2, and the other articulating joint is coupled to the mitral valve at the junction of P2 and P3. Any movement of the prosthesis at these locations is generally dispersed in the commissural space, and therefore is unlikely to tear the leaflets.

In some applications of the present invention, a surgeon independently selects the length of the posterior bar and the length of side bars, in order to provide a close fit between the prosthesis and the particular native valve. A kit is provided that includes a plurality of prostheses, which have posterior and side bars of various lengths and of various ratios of lengths. The plurality of sizes and proportions accommodates native valves having varying sizes and proportions.

For some applications, the surgeon sizes the valve prosthesis in accordance with the anterior leaflet of the native mitral valve. The surgeon measures the distance between the lateral and medial fibrous trigones of the native mitral valve, and selects the length of the posterior bar responsively to the inter-trigone distance. The surgeon measures the height of the anterior leaflet (the anterior-posterior diameter) of the native mitral valve, and selects the length of the side bars responsively to the height.

For some applications of the present invention, an annuloplasty prosthesis is coupled to a native mitral valve posterior leaflet by suturing (or otherwise coupling) the prosthesis at a plurality of locations on the prosthesis, including suturing two of the locations on the prosthesis to two sides of P2 directly opposite small commissures at (a) a junction of P1 and P2, and (b) a junction of P2 and P3, respectively. The annuloplasty prosthesis may either be a conventional prosthesis, or one of the prostheses described herein. For some applications, an inter-trigone distance between lateral and medial fibrous trigones of the mitral valve is measured, and the two locations are selected responsively to the measured inter-trigone distance, typically such that a distance between the two locations equals between 90% and 110% of the inter-trigone distance.

In some applications of the present invention, an annuloplasty kit is provided, which comprises a plurality of annuloplasty prostheses, which may be either conventional prostheses or some of the prostheses described herein. At least two of the prostheses (e.g., at least three of the prostheses) in the kit have a same side-to-side width, and different respective anterior-posterior heights. Typically, when using the kit, one or more anatomic distances of a mitral valve are measured. Responsively to the one or more measured distances, an appropriately-sized one of the prostheses is selected from the kit, and implanted at the native mitral valve. Typically, a height of an anterior leaflet of the mitral valve is measured. Typically, an inter-trigone distance between lateral and medial fibrous trigones of the mitral valve is additionally measured. Optionally, a height of a posterior leaflet of the mitral valve is additionally measured.

In some applications of the present invention, an inter-trigone distance between lateral and medial fibrous trigones of a native mitral valve is measured. An annuloplasty prosthesis is implanted at the native mitral valve such that the prosthesis restores a length of P2 to the inter-trigone distance.

In some applications of the present invention, an annuloplasty prosthesis is provided that comprises a fabric sleeve and a skeleton that is positioned within a portion of the fabric sleeve. The skeleton comprises (a) a rigid posterior bar segment, which has first and second bar ends, and (b) optionally, first and second semi-rigid posterior segments, which begin at the first and second bar ends, respectively, and extend away from a longitudinal center of the rigid posterior bar segment. The prosthesis further comprises a sleeve, which typically functions as a sewing ring for coupling the prosthesis to the native mitral valve. The sleeve comprises a flexible and substantially non-extensible fabric material, and covers the rigid posterior bar segment, and, typically, covers the entire skeleton, and optionally extends beyond the ends of the skeleton. The sleeve includes a first portion that extends at least 20 mm beyond the first bar end, and a second portion that extends at least 20 mm beyond the second bar end.

Because of this design, the annuloplasty prosthesis is nearly rigid in the anterior-posterior direction, and highly flexible in the side-to-side direction. In particular, first and second anterior ends of the sleeve are free to move in the side-to-side direction as the first and second portions of the sleeve articulate with the rigid posterior bar segment. The rigid posterior bar segment has a relatively low burden, and has some freedom of motion. This freedom of motion provides the rigid posterior bar segment with some shock absorption capability, and diffuses the bending movement, thus causing the rigid posterior bar segment to be break-resistant.

The sleeve has a plurality of visually-sensible fiducial designators, at respective longitudinal locations, typically distributed along at least 95% (e.g., 100%) of the sleeve (typically to the ends or near the ends of the sleeve). The fiducial designators typically are arranged to indicate the longitudinal center of the rigid posterior bar segment. For example, the designators may include a central posterior fiducial designator located at a longitudinal center of the rigid posterior bar segment. The central posterior fiducial designator is typically distinguished from the other fiducial designators (for example, it may be slightly darker or wider, or of a different color from, the other fiducial designators). Thus, the fiducial designators (other than the central posterior fiducial designator, if provided) are useful for measuring the distance from the rigid segment center toward the first and second anterior ends of the sleeve.

The prosthesis is typically rigid along rigid posterior bar segment, and is flexible or semi-rigid along at least 75% (e.g., at least 90% or 100%) of the first portion of the sleeve and along at least 75% (e.g., at least 90% or 100%) of the second portion of the sleeve.

The prosthesis is typically implanted at a native mitral valve such that the prosthesis restores a length of the annulus of the middle scallop (P2) of a posterior leaflet of the mitral valve to the inter-trigone distance. To implant the prosthesis, the surgeon measures an inter-trigone distance between lateral and medial fibrous trigones of the native mitral valve. Typically, the surgeon then couples (e.g., sutures, preferably pledgeted) to the center of the rigid posterior bar segment to the middle of the P2 annulus. The rigid posterior bar segment is positioned along the P2 annulus. Pairs of at least a portion of the fiducial designators on opposite sides of the longitudinal center of the rigid posterior bar segment are spaced apart at distances representative of typical inter-trigone distances. These distances may be used for identifying two locations along the sleeve for coupling to the posterior leaflet at the annulus of the native mitral valve. The surgeon selects, and optionally marks (e.g., with a needle or a pen), two locations at half of the measured inter-trigone distance on each side of the center of the sleeve, using the fiducial designators for measuring these locations on the sleeve. The surgeon couples the selected locations along the sleeve to the posterior annulus at the leaflet of the native mitral valve, using tissue anchoring elements, such as sutures (pledgeted or non-pledgeted). Typically, two primary sutures are used to couple the two locations along the sleeve, respectively, to two sides of P2 directly opposite the small commissures (minor clefts) at the junctions of P1 and P2, and P2 and P3, respectively. These primary sutures opposite the P1/P2 and P2/P3 junctions thus anchor the rigid posterior bar segment down into its exact anatomical location. (The middle suture is at the weakest point of the annulus, and is thus more susceptible to dehiscence, and it therefore may be beneficial for it to be pledgeted.)

For applications in which the skeleton comprises the first and second semi-rigid posterior segments, as described hereinabove, and the two locations are along these semi-rigid posterior segments, the rigid posterior bar segment provides complete rigidity along most of the base of the posterior leaflet, while the semi-rigid posterior segments provide partial rigidity along the remainder of the base of the posterior leaflet.

For some applications, the surgeon measures a height of the anterior leaflet (A) of the mitral valve, and, optionally, also a height of the anterior leaflet (P) of the mitral valve. Responsively to the measured height of the posterior leaflet, and, optionally, the measured height of the posterior leaflet (typically, the sum of the two heights), the surgeon ascertains a desired anterior-posterior height of the prosthesis. For some applications, the surgeon ascertains the anterior-posterior height of the prosthesis responsively to the sum of the anterior and posterior heights less a value indicative of twice a coaptation length, and typically minus the posterior bar curvature height, and/or minus an additional constant to account for the depth of coaptation in the beating heart.

For some applications, the prosthesis is implanted as a band (i.e., partial ring). For these applications, in order to achieve the desired anterior-posterior height of the prosthesis, if necessary the surgeon trims the first and second portions of the sleeve, leaving appropriate lengths of the sleeve on both sides to reach the trigones. The surgeon uses the fiducial designators to measure the length of the sleeve on both sides of the center. After any necessary trimming, the surgeon couples, e.g., sutures, the sleeve, at or near its trimmed ends, to the lateral and medial trigones, respectively.

For other applications, the prosthesis is implanted a complete ring. For these applications, in order to achieve the desired anterior-posterior height of the lateral side of the prosthesis, the surgeon trims the first portion of the sleeve, as described above. The surgeon couples the sleeve at or near the trimmed end to the lateral trigone. The surgeon couples the second portion of the sleeve at a corresponding distance from the center to the medial trigone. The surgeon estimates the length of an inter-trigone portion of the sleeve to be equal to the measured inter-trigone distance. The surgeon uses the fiducial designators to measure the distance from the location at which the sleeve is coupled to the medial fibrous trigone. The surgeon trims any excess sleeve beyond the desired location. The surgeon then couples the sleeve at or near the trimmed end of the second portion, to the mitral valve at the lateral trigone, thereby completing a complete ring.

When using the fiducial designators for measuring distances along the sleeve, the surgeon typically measures each successive distance on a given side of the sleeve from the previously-measured distance on this side, which is typically marked on the sleeve by the surgeon during the implantation procedure.

For some applications of the present invention, one goal is to provide a single type of device, which will be amenable to fit all native valve sizes and proportions, while maintaining the basic principles of providing P2 with rigid support, the size of the inter-trigone distance of the particular patient, using straightforward surgical steps. In order to provide rigid support to the weakest point of the posterior valve annulus, the center of the rigid posterior bar is typically absolutely rigid, fit for the smallest size native valve, i.e., typically 25-26 mm. Typically, on both ends the rigid posterior bar continues with semi-rigid segments, typically having a length of 4-10 mm on each side. The sum of the length of the rigid posterior bar and the semi-rigid segments provides up to 46 mm of posterior rigid support. This design allows a manufacturer to provide the annuloplasty prosthesis is a single size (“unisize” or one-size-fits-all). Nevertheless, the manufacturer may prefer to provide the annuloplasty prosthesis in sets including two or three sizes, for example for inter-trigone distances of 25-30 mm (small), 32-36 mm (medium) and 38-46 mm (large), or for small valves (25-32 mm) and large valve (34 mm and above). Typically, the only difference between the different size prostheses is the length of the rigid posterior segment, which may be equal to the smallest number in the range of inter-trigone distances for a given size (such as 25 mm in the small size, and 34 mm or 38 mm, respectively, in the large sizes mentioned above. (All the division limits between small, medium, and large, or large and small are intuitive, and generally arbitrary.)

There is therefore provided, in accordance with an application of the present invention, a method including:

providing an annuloplasty prosthesis; and

coupling the prosthesis to a native mitral valve posterior annulus at a plurality of locations on the prosthesis, including coupling two of the locations on the prosthesis to two sides of a middle scallop (P2) of the posterior annulus directly opposite small commissures at:

a junction of a lateral scallop (P1) of the posterior leaflet and P2, and

a junction of P2 and a medial scallop (P3) of the posterior leaflet, respectively.

For some applications, coupling includes suturing the prosthesis to the native mitral valve annulus.

For some applications, coupling includes measuring an inter-trigone distance between lateral and medial fibrous trigones of the mitral valve, and selecting the two locations responsively to the measured inter-trigone distance. For some applications, selecting the two locations includes selecting the two locations such that a distance between the two locations equals between 90% and 110% of the inter-trigone distance.

For some applications, the prosthesis includes a complete ring annuloplasty prosthesis. Alternatively, for some applications, the prosthesis includes a partial annuloplasty prosthesis, such as an annuloplasty band.

There is further provided, in accordance with an application of the present invention, apparatus including an annuloplasty prosthesis, which includes:

a skeleton, which includes a rigid posterior bar segment, which has first and second bar ends, and a length, measured in a straight line between the first and the second bar ends, of between 24 and 48 mm; and

a sleeve, which (a) includes a flexible and substantially non-extensible fabric material, (b) covers the rigid posterior bar segment, (c) includes a first portion that extends at least 20 mm beyond the first bar end and a second portion that extends at least 25 mm beyond the second bar end, and (d) has a plurality of visually-sensible fiducial designators at respective longitudinal locations distributed along at least a portion of the sleeve, which fiducial designators are arranged to indicate a longitudinal center of the rigid posterior bar segment,

wherein the prosthesis is rigid along the rigid posterior bar segment, and is flexible or semi-rigid along at least 75% of the first portion of the sleeve and along at least 75% of the second portion of the sleeve.

For some applications, the fiducial designators include a central posterior fiducial designator located at the longitudinal center of the rigid posterior bar segment. For some applications, the central posterior fiducial designator is distinguished from the other fiducial designators.

For some applications, the fiducial designators are distributed along at least 95% of the sleeve.

For some applications, the second portion of the sleeve extends at least 50 mm beyond the second bar end.

For some applications, the prosthesis is flexible or semi-rigid (a) between the first bar end and a posterior-most one of the first anterior fiducial designators and (b) between the second bar end and a posterior-most one of the second anterior fiducial designators.

For some applications, the prosthesis is flexible or semi-rigid at all longitudinal locations other than along the rigid posterior bar segment.

For some applications, the prosthesis is flexible at all locations (a) beyond 15 mm beyond the first bar end and (b) beyond 15 mm beyond the second bar end.

For some applications, the fiducial designators include a material woven into the sleeve. Alternatively or additional, the fiducial designators may include ink.

For some applications, the fiducial designators are equally spaced along the sleeve.

For some applications, the fiducial designators are shaped as respective lines that surround at least half of a perimeter of the sleeve at respective longitudinal locations.

For some applications, one of the fiducial designators is located longitudinally at the first bar end, and one of the fiducial designators is located longitudinally at the second bar end.

For some applications, the skeleton further includes first and second semi-rigid posterior segments, which begin at the first and the second bar ends, respectively, and extend away from a longitudinal center of the rigid posterior bar segment. For some applications, each of the first and the second semi-rigid posterior segments has a length of between 4 and 15 mm. For some applications, the skeleton includes a semi-rigid structural element, having (a) first and second portions that respectively define the first and the second semi-rigid posterior segments, and (b) a third portion that longitudinally coincides with the rigid posterior bar segment. For some applications, the rigid posterior bar segment passes through the portion of the semi-rigid structural element. For some applications, the portion of the semi-rigid structural element passes through the rigid posterior bar segment. For some applications, the skeleton further includes first and second flexible side segments, which begin at first and second anterior ends of the first and the second semi-rigid posterior segments, respectively, and extend away from the longitudinal center of the rigid posterior bar segment.

For some applications, the posterior bar is curved.

For some applications, a total length of the sleeve is between 100 and 180 mm.

For some applications, a length of the sleeve beyond the first bar end is between 20 and 50 mm, and a length of the sleeve beyond the second bar end is between 20 and 100 mm.

For some applications, a length of the sleeve beyond the second bar end is at least 20 mm greater than a length of the sleeve beyond the first bar end.

For some applications, the prosthesis further includes a plurality of tissue anchoring elements. For some applications, the tissue anchoring elements include sutures.

For some applications, the skeleton consists entirely of metal.

There is still further provided, in accordance with an application of the present invention, a method including:

measuring an inter-trigone distance between lateral and medial fibrous trigones of a native mitral valve of a subject; and

implanting an annuloplasty prosthesis at the native mitral valve such that the prosthesis restores a length of a middle scallop (P2) of a posterior leaflet of the mitral valve to the inter-trigone distance.

For some applications, implanting includes: responsively to the measured inter-trigone distance, selecting the annuloplasty prosthesis from a plurality of differently-sized annuloplasty prostheses; and implanting the selected prosthesis at the native mitral valve.

For some applications, selecting the prosthesis includes selecting the prosthesis responsively to a comparison of a length of a posterior portion of the prosthesis and the inter-trigone distance.

For some applications: the posterior portion of the prosthesis is a rigid posterior bar having first and second ends,

the prosthesis includes (a) the rigid posterior bar, and (b) non-extensible first and second side bars having first and second anterior ends, respectively, and first and second posterior ends, respectively, the first and second posterior ends of the side bars are coupled to the first and second ends of the posterior bar, respectively, such that the first and second side bars articulate with the posterior bar at first and second articulating joints, respectively,

implanting including placing the prosthesis at the native mitral valve such that the posterior bar is positioned along the middle scallop (P2), the first side bar is positioned along a lateral scallop (P1) of the posterior leaflet and extending to a lateral fibrous trigone of the mitral valve, and the second side bar is positioned along a medial scallop (P3) of the posterior leaflet and extending to a medial fibrous trigone of the mitral valve, and

selecting the prosthesis responsively to the comparison includes selecting one of the plurality of the prostheses having the rigid posterior bar having a length closest to the inter-trigone distance.

For some applications:

the prosthesis includes (a) a skeleton, which includes a rigid posterior bar segment, which has first and second bar ends, and a length of between 24 and 48 mm, and (b) a sleeve, which (i) includes a flexible and substantially non-extensible fabric material, (ii) covers the rigid posterior bar segment, and (iii) includes a first portion that extends at least 20 mm beyond the first bar end and a second portion that extends at least 20 mm beyond the second bar end, and

implanting includes coupling the prosthesis, at a plurality of locations along the sleeve, to a posterior leaflet annulus of the native mitral valve.

For some applications, coupling includes suturing the prosthesis to the posterior leaflet.

For some applications, coupling the sleeve includes:

selecting two of the locations along the sleeve that are on opposite longitudinal sides of a longitudinal center of the rigid posterior bar segment; and

coupling the sleeve at the two locations to two sides of the middle scallop (P2) of the posterior leaflet annulus directly opposite small commissures at:
a junction of a lateral scallop (P1) of the posterior leaflet and P2, and
a junction of P2 and a medial scallop (P3) of the posterior leaflet, respectively.

For some applications, coupling further includes, prior to coupling the sleeve at the two locations, coupling the sleeve at the longitudinal center of the rigid posterior bar segment to a middle of a middle scallop (P2) annulus.

For some applications, selecting the two locations includes selecting two locations that are equidistant from the center of the rigid posterior bar segment. For some applications, coupling includes selecting the two locations along the sleeve responsively to the measured inter-trigone distance. For some applications, selecting the two locations along the sleeve includes selecting the two locations along the sleeve such that a distance between the two locations equals between 90% and 110% of the inter-trigone distance.

For some applications:

the sleeve has a plurality of visually-sensible fiducial designators at respective longitudinal locations distributed along at least a portion of the sleeve, which fiducial designators are arranged to indicate a longitudinal center of the rigid posterior bar segment, and

selecting the two locations along the sleeve includes selecting the two locations along the sleeve using at least two of the fiducial designators.

For some applications, the fiducial designators are distributed along at least 95% of the sleeve.

For some applications, the prosthesis is rigid along the rigid posterior bar segment, and is flexible or semi-rigid along at least 75% of the first portion of the sleeve and along at least 75% of the second portion of the sleeve.

For some applications, the method further includes:

measuring a height of an anterior leaflet of the native mitral valve; and

responsively to the measured height, trimming the first and the second portions of the sleeve if necessary.

For some applications, the method further includes measuring a height of a posterior leaflet of the native mitral valve, and trimming includes trimming responsively to both the measured height of the anterior leaflet and the measured height of the posterior leaflet.

For some applications, the sleeve has a plurality of visually-sensible fiducial designators at respective longitudinal locations distributed along at least 95% of the sleeve, and trimming includes trimming the first and the second portions of the sleeve responsively to the measured height using at least two of the fiducial designators. For some applications, implanting the prosthesis includes, after trimming the first portion, coupling the first portion of the sleeve at or near an anterior end thereof to the mitral valve at a first one of the fibrous trigones. For some applications, implanting the prosthesis includes, after trimming the second portion, coupling the second portion of the sleeve at or near an anterior end thereof to the mitral valve at a second one of the fibrous trigones. For some applications, trimming the second end portion includes trimming the second end portion responsively to both the measured height and the inter-trigone distance, such that the second portion, after being trimmed, is longer than the first portion, after being trimmed. For some applications, implanting the prosthesis includes, after trimming the second portion, coupling the second portion of the sleeve at or near an anterior end thereof to the mitral valve at the first one of the fibrous trigones.

For some applications, the prosthesis is flexible or semi-rigid at all longitudinal locations other than along the rigid posterior bar segment.

For some applications, the prosthesis is flexible at all locations (a) beyond 15 mm beyond the first bar end and (b) beyond 15 mm beyond the second bar end.

For some applications, the sleeve has a plurality of visually-sensible fiducial designators at respective longitudinal locations, which fiducial designators are arranged to indicate a longitudinal center of the rigid posterior bar segment, and implanting includes coupling the sleeve at the longitudinal center of the rigid posterior bar segment to a posterior annulus of the native mitral valve.

For some applications, the skeleton further includes first and second semi-rigid posterior segments, which begin at the first and the second bar ends, respectively, and extend away from a longitudinal center of the rigid posterior bar segment. For some applications, each of the first and the second semi-rigid posterior segments has a length of between 4 and 15 mm.

For some applications, the posterior bar is curved.

For some applications, a total length of the sleeve is between 100 and 180 mm.

For some applications, a length of the sleeve beyond the first bar end is between 20 and 70 mm, and a length of the sleeve beyond the second bar end is between 20 and 100 mm.

There is additionally provided, in accordance with an application of the present invention, a method including:

providing an annuloplasty prosthesis that includes (a) a skeleton, which includes a rigid posterior bar segment, which has first and second bar ends, and a length, measured in a straight line between the first and the second bar ends, of between 24 and 48 mm, and (b) a sleeve, which (i) includes a flexible and substantially non-extensible fabric material, (ii) covers the rigid posterior bar segment, and (iii) includes a first portion that extends at least 20 mm beyond the first bar end and a second portion that extends at least 20 mm beyond the second bar end;

measuring an inter-trigone distance between lateral and medial fibrous trigones of a native mitral valve of a subject;

responsively to the measured inter-trigone distance, selecting two locations along the sleeve that are on opposite longitudinal sides of a longitudinal center of the rigid posterior bar segment; and

implanting the annuloplasty prosthesis at the native mitral valve by coupling the prosthesis to a posterior leaflet of the native mitral valve, including at the two locations along the sleeve.

For some applications, selecting the two locations includes selecting two locations that are equidistant from the center of the rigid posterior bar segment.

For some applications, coupling includes suturing the prosthesis.

For some applications, coupling the sleeve includes coupling the sleeve at the two of the locations along the sleeve to two sides of a middle scallop (P2) of the posterior leaflet directly opposite small commissures at:
a junction of a lateral scallop (P1) of the posterior leaflet and P2, and
a junction of P2 and a medial scallop (P3) of the posterior leaflet, respectively.

For some applications, coupling further includes, prior to coupling the sleeve at the two locations, coupling the sleeve at a longitudinal center of the rigid posterior bar segment to a middle of the P2 annulus.

For some applications, selecting the two locations along the sleeve includes selecting the two locations along the sleeve such that a distance between the two locations equals between 90% and 110% of the inter-trigone distance.

For some applications, the sleeve has a plurality of visually-sensible fiducial designators at respective longitudinal locations distributed along at least a portion of the sleeve, and selecting the two locations along the sleeve includes selecting the two locations along the sleeve using at least two of the fiducial designators.

For some applications, the fiducial designators are distributed along at least 95% of the sleeve.

For some applications, the prosthesis is rigid along the rigid posterior bar segment, and is flexible or semi-rigid along at least 75% of the first portion of the sleeve and along at least 75% of the second portion of the sleeve.

For some applications, the method further includes: measuring a height of an anterior leaflet of the native mitral valve; and responsively to the measured height, trimming the first and the second portions of the sleeve if necessary.

For some applications, the method further includes measuring a height of a posterior leaflet of the native mitral valve, and trimming includes trimming responsively to both the measured height of the anterior leaflet and the measured height of the posterior leaflet.

For some applications, the sleeve has a plurality of fiducial designators at respective longitudinal locations distributed along at least 95% of the sleeve, and trimming includes trimming the first and the second portions of the sleeve responsively to the measured height using at least two of the fiducial designators. For some applications, implanting the prosthesis includes, after trimming the first portion, coupling the first portion of the sleeve at or near an anterior end thereof to the mitral valve at a first one of the fibrous trigones. For some applications, implanting the prosthesis includes, after trimming the second portion, coupling the second portion of the sleeve at or near an anterior end thereof to the mitral valve at a second one of the fibrous trigones. For some applications, trimming the second end portion includes trimming the second end portion responsively to both the measured height and the inter-trigone distance, such that the second portion, after being trimmed, is longer than the first portion, after being trimmed. For some applications, implanting the prosthesis includes, after trimming the second portion, coupling the second portion of the sleeve at or near an anterior end thereof to the mitral valve at the first one of the fibrous trigones.

For some applications, the prosthesis is flexible or semi-rigid (a) between the first bar end and a posterior-most one of the first anterior fiducial designators and (b) between the second bar end and a posterior-most one of the second anterior fiducial designators.

For some applications, the prosthesis is flexible or semi-rigid at all longitudinal locations other than along the rigid posterior bar segment.

For some applications, the prosthesis is flexible at all locations (a) beyond 15 mm beyond the first bar end and (b) beyond 15 mm beyond the second bar end.

For some applications, the sleeve has a plurality of visually-sensible fiducial designators at respective longitudinal locations, which fiducial designators are arranged to indicate a longitudinal center of the rigid posterior bar segment, and implanting includes coupling the sleeve at the longitudinal center of the rigid posterior bar segment to a posterior annulus of the native mitral valve.

For some applications, the skeleton further includes first and second semi-rigid posterior segments, which begin at the first and the second bar ends, respectively, and extend away from a longitudinal center of the rigid posterior bar segment. For some applications, each of the first and the second semi-rigid posterior segments has a length of between 4 and 15 mm.

For some applications, the posterior bar is curved.

For some applications, a total length of the sleeve is between 100 and 180 mm.

For some applications, a length of the sleeve beyond the first bar end is between 20 and 70 mm, and a length of the sleeve beyond the second bar end is between 20 and 100 mm.

There is yet additionally provided, in accordance with an application of the present invention, a method including:

positioning a pledget and one or more sutures such that (a) the pledget is placed against a ventricular side of a native mitral valve below a middle scallop (P2) of a posterior leaflet of the mitral valve at a center of a posterior portion of a mitral annulus, and (b) the one or more sutures pass through the pledget and the posterior leaflet at the mitral annulus such that portions of the sutures extend into the atrium; and

using the portions of the one or more sutures that extend into the atrium, coupling an annuloplasty prosthesis to the posterior leaflet at the mitral annulus.

For some applications, positioning the one or more pledgeted sutures from below the ring includes avoiding capturing chordae tendineae with the sutures.

There is also provided, in accordance with an application of the present invention, a method including:

introducing a catheter into a left ventricle;

using the catheter, introducing an anchor that includes an anchor base fixed to at least one elongated anchoring member;

coupling the anchor base to a ventricular side of a posterior leaflet of a native mitral valve below a middle scallop (P2) of the posterior leaflet at a center of a posterior portion of a mitral annulus;

passing the at least one elongated anchoring member through the posterior leaflet into a left atrium; and

using the elongated anchoring element, coupling an annuloplasty prosthesis to the mitral annulus.

For some applications, the at least one elongated anchoring member is at least one wire.

For some applications, the anchor base is an umbrella anchor, and coupling the anchor base includes expanding the umbrella anchor against the ventricular side of the posterior leaflet, at the mitral annulus.

There is further provided, in accordance with an application of the present invention, apparatus including an annuloplasty prosthesis having at least first and second visually-sensible fiducial designators at respective first and second locations on the prosthesis, wherein the first fiducial designator is at between 7 and 8 o'clock, and the second fiducial designator is at between 4 and 5 o'clock, assuming that 6 o'clock is at a center of a posterior portion of the prosthesis.

For some applications, the prosthesis includes a complete annuloplasty prosthesis. Alternatively, the prosthesis includes a partial annuloplasty prosthesis, such as an annuloplasty band.

There is still further provided, in accordance with an application of the present invention, a method including:

providing an annuloplasty prosthesis having at least first and second visually-sensible fiducial designators at respective first and second locations on the prosthesis, wherein the first fiducial designator is at between 7 and 8 o'clock, and the second fiducial designator is at between 4 and 5 o'clock, assuming that 6 o'clock is at a center of a posterior portion of the prosthesis; and

coupling the prosthesis to a native mitral valve posterior annulus leaflet by coupling the prosthesis at the first and second locations to the mitral valve.

For some applications, coupling includes suturing the prosthesis to the mitral valve.

For some applications, coupling includes coupling the prosthesis at the first and second locations to two side of a middle scallop (P2) of the posterior leaflet directly opposite small commissures at (a) a junction of a lateral scallop (P1) of the posterior leaflet and P2, and (b) a junction of P2 and a medial scallop (P3) of the posterior leaflet, respectively.

For some applications, the annuloplasty prosthesis is one of a plurality of annuloplasty prostheses having different sizes, and providing the prosthesis includes:

providing a kit including the plurality of annuloplasty prostheses;

measuring an inter-trigone distance between lateral and medial fibrous trigones of the mitral valve; and

selecting one of the annuloplasty prostheses as the annuloplasty prosthesis based on a comparison of the inter-trigone distance and a distance between the first and second fiducial designators.

For some applications, selecting includes selecting the one of the annuloplasty prostheses having the distance between the first and second fiducial designators that is closest to the inter-trigone distance.

For some applications, the prosthesis is a complete annuloplasty prosthesis. Alternatively, the prosthesis is a partial annuloplasty prosthesis, such as an annuloplasty band.

There is additionally provided, in accordance with an application of the present invention, apparatus including an annuloplasty prosthesis, which includes a skeleton, which includes:

a rigid posterior bar, which has first and second ends, and a length of between 20 and 50 mm; and

non-extensible first and second side bars, which have first and second anterior ends, respectively, and first and second posterior ends, respectively, and respective lengths of between 25 and 45 mm,

wherein the first and second posterior ends of the side bars are coupled to the first and second ends of the posterior bar, respectively, such that the first and second side bars articulate with the posterior bar, and the first and second anterior ends are not coupled to any elements of the skeleton.

For some applications, the posterior bar and first and second side bars are curved.

For some applications, the prosthesis further includes a fabric covering that covers the skeleton.

For some applications, the fabric covering is shaped so as define a closed ring.

For some applications, the fabric covering includes: a covering segment, in which the skeleton is positioned; and a connecting segment, in which the skeleton is not positioned, and which extends between the first and second anterior ends of the first and second side bars, respectively. For some applications, a length of the connecting segment is equal to between 80% and 120% of the length of the posterior bar.

For some applications, the prosthesis further includes a plurality of tissue anchoring elements. For some applications, the prosthesis includes at least eight tissue anchor elements. For some applications, the tissue anchoring elements include sutures.

For some applications:

the first and second posterior ends of the side bars are shaped so as to define first and second side bar rings, respectively,

the first and second ends of the posterior bar are shaped so as to define first and second posterior bar rings, respectively, and

the first and second posterior bar rings are coupled to the first and second side bar rings, respectively, such that the first and second side bars articulate with the posterior bar.

For some applications:

the posterior bar is shaped so as to define a curve that defines a posterior bar plane,

the first and second posterior bar rings define first and second posterior bar ring planes, respectively, and

a first angle between the posterior bar plane and the first posterior bar ring plane, and a second angle between the posterior bar plane and the second posterior bar ring plane, are both less than 30 degrees.

For some applications, the first and second angles are both less than 5 degrees.

For some applications:

the first side bar is shaped so as to define a curve that defines a side bar plane,

the first bar ring defines a first side bar ring plane, and

an angle between the side bar plane and the first side bar ring plane is between 60 and 120 degrees.

For some applications, the angle is between 85 and 95 degrees.

For some applications, the skeleton consists entirely of metal.

For some applications, the skeleton consists of only the posterior bar and the first and second side bars.

There is yet additionally provided, in accordance with an application of the present invention, apparatus including an annuloplasty kit, which includes at least four annuloplasty prostheses, each of which includes:
a rigid posterior bar, which has first and second ends, and a length of between 20 and 50 mm; and
non-extensible first and second side bars, which have first and second posterior ends, respectively, and respective lengths of between 25 and 45 mm,
wherein the first and second posterior ends of the side bars are coupled to the first and second ends of the posterior bar, respectively, such that the first and second side bars articulate with the posterior bar,

wherein at least two of the annuloplasty prostheses have respective posterior bars having the same length, and respective first side bars having different lengths.

For some applications, at least two of the annuloplasty prostheses have respective first side bars having the same length, and respective posterior bars having different lengths.

For some applications, each of the annuloplasty prostheses further includes a fabric covering that is configured to cover the posterior bar and first and second side bars thereof.

For some applications, each of the annuloplasty prostheses further includes a plurality of tissue anchoring elements. For some applications, the tissue anchoring elements include sutures.

There is also provided, in accordance with an application of the present invention, a method including:

providing an annuloplasty prosthesis, which includes (a) a rigid posterior bar having first and second ends, and (b) non-extensible first and second side bars having first and second anterior ends, respectively, and first and second posterior ends, respectively, wherein the first and second posterior ends of the side bars are coupled to the first and second ends of the posterior bar, respectively, such that the first and second side bars articulate with the posterior bar at first and second articulating joints, respectively; and

placing the prosthesis at a native mitral valve, such that the posterior bar is positioned along a middle scallop (P2) of a posterior leaflet of the mitral valve, the first side bar is positioned along a lateral scallop (P1) of the posterior leaflet and extending to a lateral fibrous trigone of the mitral valve, and the second side bar is positioned along a medial scallop (P3) of the posterior leaflet and extending to a medial fibrous trigone of the mitral valve.

For some applications, placing the prosthesis includes coupling the first articulating joint to the mitral valve at a junction of P1 and P2, and the second articulating joint to the mitral valve at a junction of P2 and P3.

For some applications, placing the prosthesis includes coupling the first anterior end of the first side bar to a lateral fibrous trigone of the mitral valve, and the second anterior end of the second side bar to a medial fibrous trigone of the mitral valve.

For some applications, the posterior bar and first and second side bars are curved, and providing the prosthesis including providing the prosthesis having the curved bars.

For some applications, providing the prosthesis includes providing the prosthesis including a skeleton that includes the posterior bar and the first and second side bars, the prosthesis configured such that the first and second anterior ends are not coupled to any elements of the skeleton.

For some applications, the annuloplasty prosthesis is one of a plurality of annuloplasty prostheses having posterior bars of at least two different lengths, and providing the prosthesis includes:

providing a kit including the plurality of annuloplasty prostheses;

measuring an inter-trigone distance between lateral and medial fibrous trigones of the mitral valve; and

selecting one of the annuloplasty prostheses as the annuloplasty prosthesis based on a comparison of the inter-trigone distance and the lengths of the posterior bars of the annuloplasty prostheses.

For some applications, selecting includes selecting the one of the annuloplasty prostheses having a posterior bar having a length closest to the inter-trigone distance.

For some applications, selecting includes selecting the one of the annuloplasty prostheses having a posterior bar having a length equal to between 80% and 120% of the inter-trigone distance.

For some applications, the annuloplasty prosthesis is one of a plurality of annuloplasty prostheses having first side bars of at least two different lengths, and providing the prosthesis includes:

providing a kit including the plurality of annuloplasty prostheses;

measuring a height of an anterior leaflet of the mitral valve; and

selecting one of the annuloplasty prostheses as the annuloplasty prosthesis based on a comparison of the anterior leaflet height and the lengths of the first side bars of the annuloplasty prostheses.

For some applications, selecting includes selecting the one of the annuloplasty prostheses having a first side bar having a length closest to the anterior leaflet height.

For some applications, the posterior bars are curved and have respective posterior bar curvature heights, and selecting includes selecting the one of the annuloplasty prostheses having a first side bar having a length closest to the anterior leaflet height less the posterior bar curvature height of the annuloplasty prosthesis.

There is further provided, in accordance with an application of the present invention, a method including:

providing a kit including at least four annuloplasty prostheses, each of which includes (a) a rigid posterior bar, which has first and second ends, and (b) non-extensible first and second side bars, which have first and second anterior ends, respectively, and first and second posterior ends, respectively, wherein the first and second posterior ends of the side bars are coupled to the first and second ends of the posterior bar, respectively, such that the first and second side bars articulate with the posterior bar, and wherein at least two of the annuloplasty prostheses have respective posterior bars having the same length, and respective first side bars having different lengths;

measuring a first anatomical distance between a first set of two points of a native cardiac valve of a subject, and a second anatomical distance between a second set of two points of the native cardiac valve, the first set of points being different from the second set of points;

selecting one of the annuloplasty prostheses responsively to both (a) a first comparison of the first anatomical distance and the lengths of the posterior bars, and to (b) a second comparison of the second anatomical distance to the lengths of the side bars; and

implanting the selected prosthesis at a native cardiac valve.

For some applications, the native cardiac valve is a mitral valve, and measuring the first anatomical distance includes measuring an inter-trigone distance between lateral and medial fibrous trigones of the mitral valve.

For some applications, the native cardiac valve is a mitral valve, and measuring the second anatomical distance includes measuring a height of an anterior leaflet of the mitral valve.

For some applications, the method further includes measuring a third anatomical distance between a third set of two points of the native cardiac valve, the third set of points being different from the first set of points and from the second set of points, the second comparison is of a combination of the second and third anatomical distances to the lengths of the side bars, and selecting includes selecting one of the annuloplasty prostheses responsively to (a) the first comparison and (b) the second comparison. For some applications, the native cardiac valve is a mitral valve, and measuring the second and third anatomical distances includes measuring a height of an anterior leaflet of the mitral valve and a height of a posterior leaflet of the mitral valve, respectively.

For some applications, the kit includes at least nine annuloplasty prostheses.

For some applications, at least two of the annuloplasty prostheses have respective first side bars having the same length, and respective posterior bars having different lengths.

There is still further provided, in accordance with an application of the present invention, apparatus including an annuloplasty kit, which includes a plurality of annuloplasty prostheses, at least two of which prostheses have a same side-to-side width, and different respective anterior-posterior heights.

For some applications, the prostheses include complete annuloplasty prostheses.

For some applications, the prostheses include partial annuloplasty prostheses, such as annuloplasty bands.

For some applications, the at least two of the prostheses includes at least three of the prostheses, which have the same width, and different respective heights.

For some applications, the at least two of the prostheses are a first at least two of the prostheses, the width is a first width, and at least a second two of the prostheses, different from the first at least two of the prostheses, have a same second side-to-side width different from the first width, and different respective anterior-posterior heights.

There is additionally provided, in accordance with an application of the present invention, a method including:

providing an annuloplasty kit, which includes a plurality of annuloplasty prostheses, at least two of which prostheses have a same side-to-side width, and different respective anterior-posterior heights;

measuring one or more anatomic distances of a mitral valve of a subject;

responsively to the one or more measured distances, selecting an appropriately-sized one of the prostheses from the kit;

implanting the selected annuloplasty prosthesis at the native mitral valve.

For some applications, measuring the one or more anatomic distances includes measuring a height of an anterior leaflet of the mitral valve.

For some applications, measuring the one or more anatomic distances further includes measuring an inter-trigone distance between lateral and medial fibrous trigones of the mitral valve.

For some applications, providing the kit includes providing the kit in which the at least two of the prostheses includes at least three of the prostheses, which have the same width, and different respective heights.

For some applications, providing the kit includes providing the kit in which: the at least two of the prostheses are a first at least two of the prostheses, the width is a first width, and at least a second two of the prostheses, different from the first at least two of the prostheses, have a same second side-to-side width different from the first width, and different respective anterior-posterior heights.

There is yet additionally provided, in accordance with an application of the present invention, a method including:

measuring a height of an anterior leaflet of a native mitral valve of a subject;

responsively to the measured height, selecting an appropriately-sized annuloplasty prosthesis from a plurality of differently-sized annuloplasty prostheses; and

implanting the selected annuloplasty prosthesis at the native mitral valve.

For some applications, the method further includes measuring a height of a posterior leaflet of the native mitral valve, and selecting includes selecting the appropriately-sized annuloplasty prosthesis responsively to both the measured height of the anterior leaflet and the measured height of the posterior leaflet.

For some applications, selecting includes selecting the appropriately-sized annuloplasty prosthesis based on a comparison of respective anterior-posterior heights of the annuloplasty prostheses with a combination of the measured height of the anterior leaflet and the measured height of the posterior leaflet.

For some applications, implanting includes implanting the selected annuloplasty prosthesis at the native mitral valve without reducing the height of the posterior leaflet.

For some applications:

measuring the height of the anterior leaflet includes stretching a posterior portion of the anterior leaflet in a posterior direction, and measuring the height of the stretched anterior leaflet,

measuring the height of the posterior leaflet includes stretching an anterior portion of the posterior leaflet in an anterior direction, and measuring the height of the stretched posterior leaflet, and

selecting includes selecting the appropriately-sized annuloplasty prosthesis responsively to a comparison of respective anterior-poster heights of the annuloplasty prostheses with a sum of the measured height of the anterior leaflet and the measured height of the posterior leaflet, less a value.

For some applications, the value is between 1.2 and 2.5 cm. For some applications, the value is between 1.5 and 3 cm, in order to take into account the coaptation point depth, as described hereinbelow.

For some applications, the value equals approximately 2 times a coaptation length of the anterior and posterior leaflets, plus, optionally, a value to correct for the coaptation point depth.

For some applications, the method further includes measuring an inter-trigone distance between lateral and medial fibrous trigones of the native mitral valve, and selecting includes selecting the appropriately-sized annuloplasty prosthesis responsively to both the measured height and the measured inter-trigone distance.

For some applications, the plurality of differently-sized annuloplasty prostheses include at least two prostheses that have a same side-to-side width, and different respective anterior-posterior heights.

The present invention will be more fully understood from the following detailed description of applications thereof, taken together with the drawings, in which:

### BRIEF DESCRIPTION OF THE DRAWINGS

FIGS. 1A and 1B are schematic illustrations of an annuloplasty prosthesis, in accordance with an application of the present invention;

FIGS. 2A-G are schematic illustrations of the annuloplasty prosthesis of FIGS. 1A and 1B implanted at a native mitral valve, in accordance with an application of the present invention;

FIGS. 3A-D are schematic illustrations of techniques for selecting bar lengths of the annuloplasty prosthesis responsively to anatomical measurements, in accordance with respective applications for the present invention;

FIG. 4 is a schematic illustration of potential movements of bars of the annuloplasty prosthesis of FIGS. 1A-B, in accordance with an application of the present invention;

FIG. 5 is a schematic illustration of an annuloplasty prosthesis, in accordance with an application of the present invention;

FIGS. 6A-C are schematic illustration of differing exemplary leaflet anatomies;

FIGS. 7A-C show schematic side-views of the leaflets and a left ventricle, in accordance with an application of the present invention;

FIGS. 8A and 8B are schematic top- and side-views of the leaflets and the left ventricle, in accordance with an application of the present invention;

FIG. 8C shows a schematic side-view of the leaflets and the left ventricle, in accordance with an application of the present invention;

FIGS. 9A-D, 10A-C, and 11A-F schematically illustrate a method for pledgeting an annuloplasty prosthesis to a mitral valve, in accordance with an application of the present invention; and

FIGS. 12A-E and 13 schematically illustrate a minimally-invasive method for pledgeting an annuloplasty prosthesis to a mitral valve, in accordance with an application of the present invention;

FIGS. 14A and 14B are schematic illustrations of another annuloplasty prosthesis, in accordance with an application of the present invention

FIGS. 15A-G are schematic illustrations of a method for implanting the annuloplasty prosthesis of FIGS. 14A and 14B, in accordance with an application of the present invention; and

FIGS. 16A-B are schematic illustrations of a plication plasty technique for creating a fold in a posterior mitral valve leaflet, in accordance with an application of the present invention.

### DETAILED DESCRIPTION OF APPLICATIONS

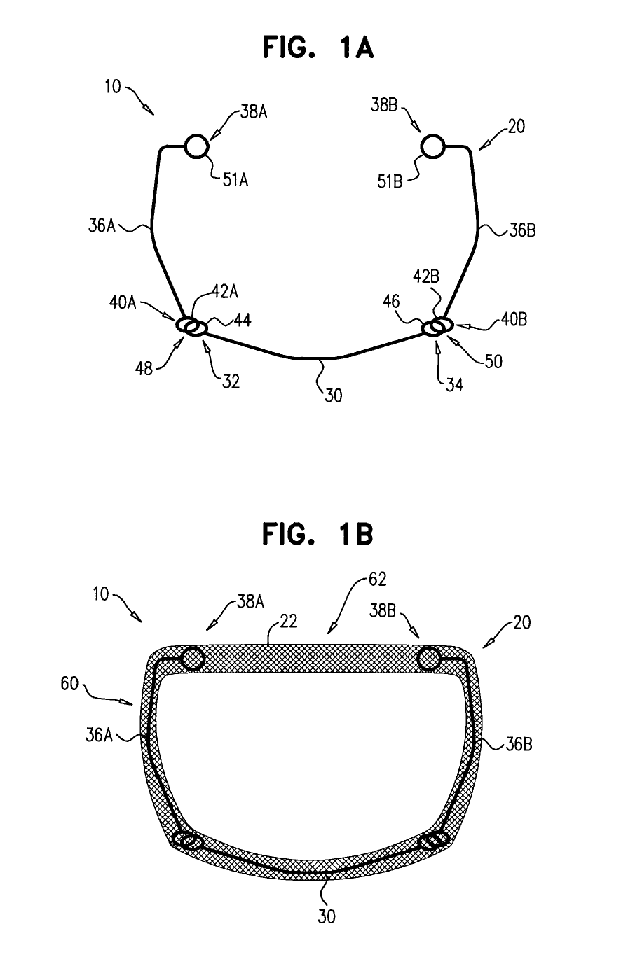

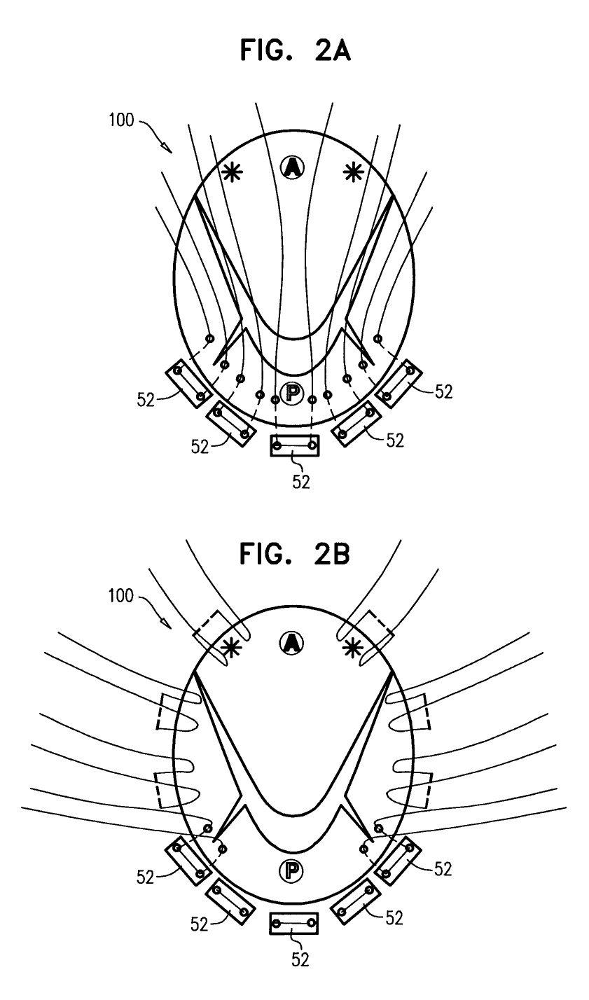

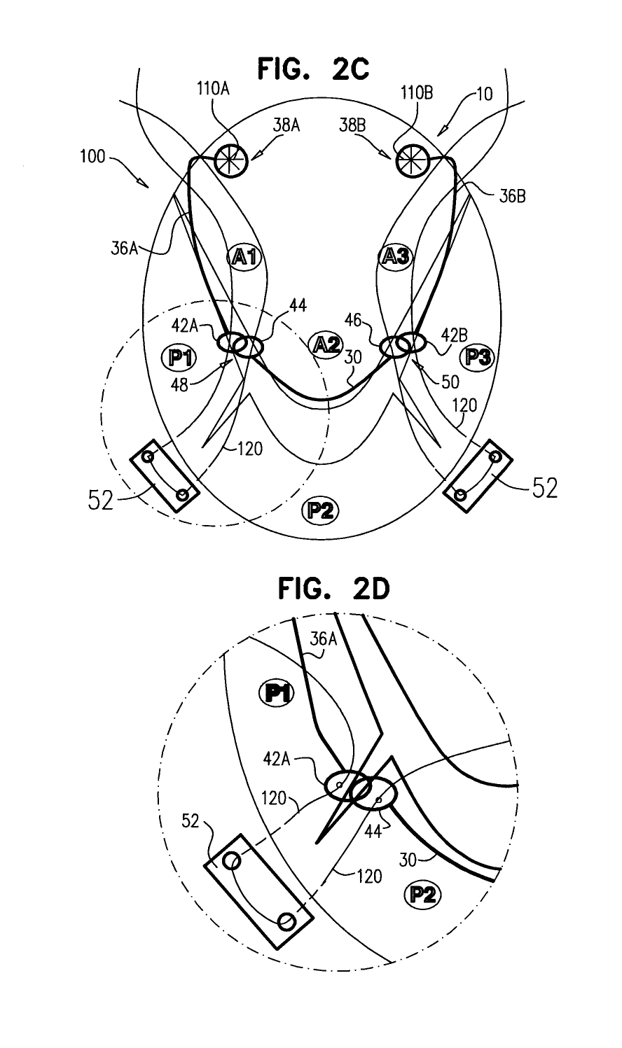

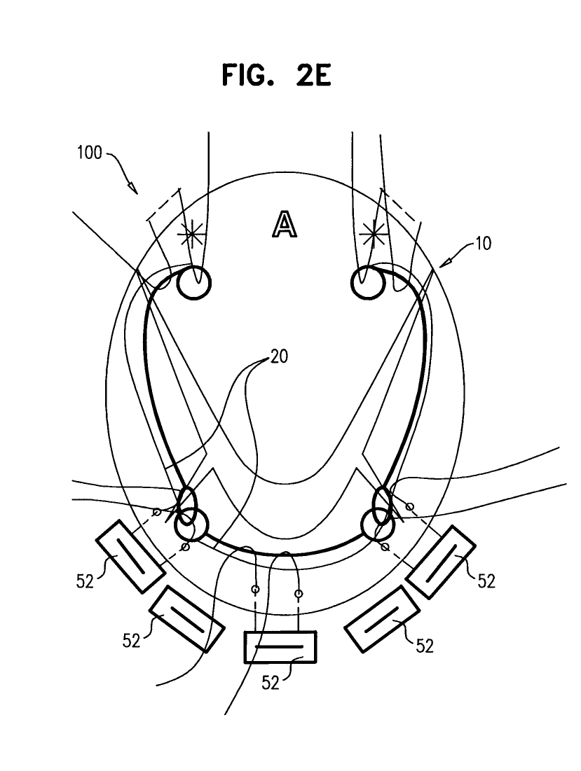

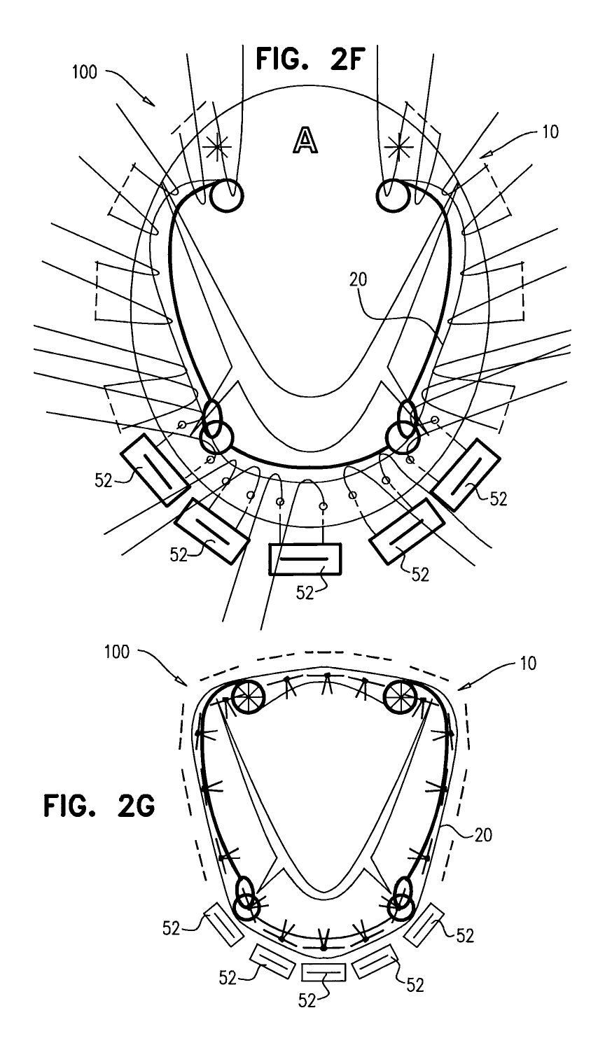

FIGS. 1A and 1B are schematic illustrations of an annuloplasty prosthesis 10, in accordance with an application of the present invention. FIG. 1A shows a skeleton 20 of the prosthesis, while FIG. 1B additionally shows in cross section a fabric covering 22 that covers the skeleton. Prosthesis 10 is configured to be placed at a native mitral valve, such as described hereinbelow with reference to FIGS. 2A-G and 3A-D.

Skeleton 20 comprises:
a rigid posterior bar 30, which has a first (lateral) end 32 and a second (medial) end 34;
a non-extensible first (lateral) side bar 36A, which has a first anterior end 38A and a first posterior end 40A; and
a non-extensible second (medial) side bar 36B, which has a second anterior end 38B and a second posterior end 40B.

First and second posterior ends 40A and 40B of the side bars are coupled to first and second ends 32 and 34 of the posterior bar, respectively, such that the first and second side bars articulate with the posterior bar. For some applications, to enable this articulation, first and second posterior ends 40A and 40B of the side bars are shaped so as to define first and second posterior side bar rings 42A and 42B, respectively. First and second ends 32 and 34 of the posterior bar are shaped so as to define first and second posterior bar rings 44 and 46, respectively. First and second posterior bar rings 44 and 46 are coupled to first and second posterior side bar rings 42A and 42B, respectively, such that the first and second side bars articulate with the posterior bar at first and second articulating joints 48 and 50, respectively. Typically, the rings are linked to one another like the links of a conventional metal chain. For some applications, the rings are elliptical, such as circular; for example, each of the rings may have an external diameter of at least 4 mm, no more than 8 mm, and/or between 4 and 8 mm. Skeleton 20 typically assumes a generally horseshoe- or C-shape, albeit with flexibility at articulating joints 48 and 50.

First and second side bars 36A and 36B are non-extensible, i.e., their respective lengths are fixed, and the bars cannot stretch or longitudinally contract. The first and second side bars may be rigid, flexible, or semi-rigid, or have different respective rigidities.

Typically, the elements of skeleton 20 articulate with one another only at first and second articulating joints 48 and 50. For some applications, skeleton 20 consists of only posterior bar 30 and first and second side bars 36A and 36B, and no other elements.

As used in the present application, including in the claims, “rigid” means stiff and resisting bending. Skeleton 20, including the posterior and side bars, typically comprises entirely metal, such as titanium, or a metal alloy. Alternatively, all or a portion of the skeleton comprises a non-metal material, such as a synthetic resin or a plastic.

For some applications, first and second anterior ends 38A and 38B of the side bars are shaped so as to define first and second anterior side bar rings 51A and 51B, respectively. These rings are typically used to couple the anterior ends of the bars to the fibrous trigones of the mitral valve, as described hereinbelow with reference to FIGS. 2A-G. For some applications, the rings are elliptical, such as circular; for example, each of the rings may have a diameter of at least 4 mm, no more than 6 mm, and/or between 4 and 6 mm.

Typically, first and second anterior ends 38A and 38B of the side bars are not coupled to any elements of skeleton 20. (It is noted in this context that anterior bar rings 51A and 51B are defined by anterior ends 38A and 38B, and are thus not considered separate elements of skeleton 20.)

Because of this design, skeleton 20 and annuloplasty prosthesis 10 are absolutely rigid in the anterior-posterior direction, and highly flexible in the side-to-side direction. In particular, anterior ends 38A and 38B of the side bars are free to move in the side-to-side direction as the side bars articulate with the posterior bar at articulating joints 48 and 50. Posterior bar 30 has a relatively low burden, and has some freedom of motion (like the movement of rings in a chain, because the posterior bar is suspended by rings on both ends). This freedom of motion provides the posterior bar with some shock absorption capability, and diffuses the bending movement, thus causing the posterior bar to be break-resistant.

For some applications, posterior bar 30 and first and second side bars 36A and 36B are curved. Together the bars thus are shaped so as to define a partial ring that is somewhat C-shaped, U-shaped, or horseshoe-shaped.

For some applications, posterior bar 30 has a length of at least 20 mm, no more than 50 mm, and/or between 20 and 50 mm, such as at least 25 mm, no more than 40 mm, and/or between 25 and 40 mm, and each of side bars 36A and 36B has a length of at least 25 mm, no more than 50 mm, and/or between 25 and 50 mm, such as at least 24 mm, no more than 45 mm, and/or between 25 and 45 mm. For applications in which the bars are curved, the length of each bar is measured in a straight line between the ends of the bar, rather than along the curve of the bar.

For some applications, posterior bar 30 is shaped so as to define a curve that defines a posterior bar plane, and first and second posterior bar rings 44 and 46 define first and second posterior bar ring planes, respectively. A first angle is defined between the posterior bar plane and the first posterior bar ring plane, and a second angle is defined between the posterior bar plane and the second posterior bar ring plane. Both the first and second angles are less than 30 degrees, such as less than 5 degrees, e.g., 0 degrees (i.e., the planes are parallel to and coincide with one another).

For some applications, such as in combination with the applications described in the preceding paragraph, first and second side bars 36A and 36B are shaped so as to define respective curves that define respective first and second side bar planes, and first and second posterior side bar rings 42A and 42B define respective first and second side bar ring plane. A first angle is defined between the side bar plane and the first side bar ring plane, and a second angle is defined between the side bar plane and the second side bar ring plane. Both the first and second angles are between 60 and 120 degrees, such as between 85 and 95 degrees, e.g., 90 degrees.

Alternatively, for some applications, the first angle between the posterior bar plane and the first posterior bar ring plane, and the second angle between the poster bar plane and the second posterior bar ring plane are each between 60 and 120 degrees, such as between 85 and 95 degrees, e.g., 90 degrees. Alternatively or additionally, for some applications, the first angle between the side bar plane and the first side bar ring plane, and the second angle between the side bar plane and the second side bar ring plane are each less than 30 degrees, such as less than 5 degrees, e.g., 0 degrees (i.e., the planes are parallel to and coincide with one another).

For some applications, before implantation of the prosthesis, if the prosthesis were to be placed on a flat surface, the posterior bar plane and first and second side bar planes would generally coincide. Thus, for some applications, respective pairs of the posterior side bar rings and the posterior rings would be orientated at right angles, or approximately right angles (e.g., between 60 and 120 degrees, such as between 85 and 105 degrees), to each other. After implantation, the orientation of the elements changes during the cardiac cycle.

Reference is again made to FIG. 1B. As mentioned above, for some applications annuloplasty prosthesis comprises fabric covering 22 that covers skeleton 20. Fabric covering 22 is typically shaped so as define a closed ring, as shown in the figure. Alternatively, the fabric covering is shaped so as to define a partial (open) ring (configuration not shown); for example, the fabric covering may not extend beyond anterior ends 38A and 38B of the side bars, and thus only cover skeleton 20. The fabric covering may function as a sewing ring for coupling the prosthesis to the native mitral valve. For example, the fabric covering may comprise Dacron® or Teflon®.

For some applications, fabric covering 22 comprises a covering segment 60, in which skeleton 20 is positioned, and a connecting segment 62, in which the skeleton is not positioned. Connecting segment 62 extends between first and second anterior ends 38A and 38B of the first and second side bars, respectively. Typically, a length of connecting segment 62 is approximately equal to the length of posterior bar 30, e.g., between 80% and 120%, such as between 90% and 110% of the length of the posterior bar. Alternatively, connecting segment 62 is not provided. Further alternatively, the connecting segment is provided, and the surgeon may cut it down if desired.

Reference is now made to FIGS. 2A-G, which are schematic illustrations of annuloplasty prosthesis 10 implanted at a native mitral valve 100, in accordance with respective applications of the present invention. It is noted that the mitral valve is shown highly schematically. In addition, for clarity of illustration, fabric covering 20 of prosthesis 10 is not shown in FIGS. 2C and 2D, although the fabric covering is typically provided, as shown in FIG. 1B. FIG. 2D is an enlarged view of the circled portion of FIG. 2C.

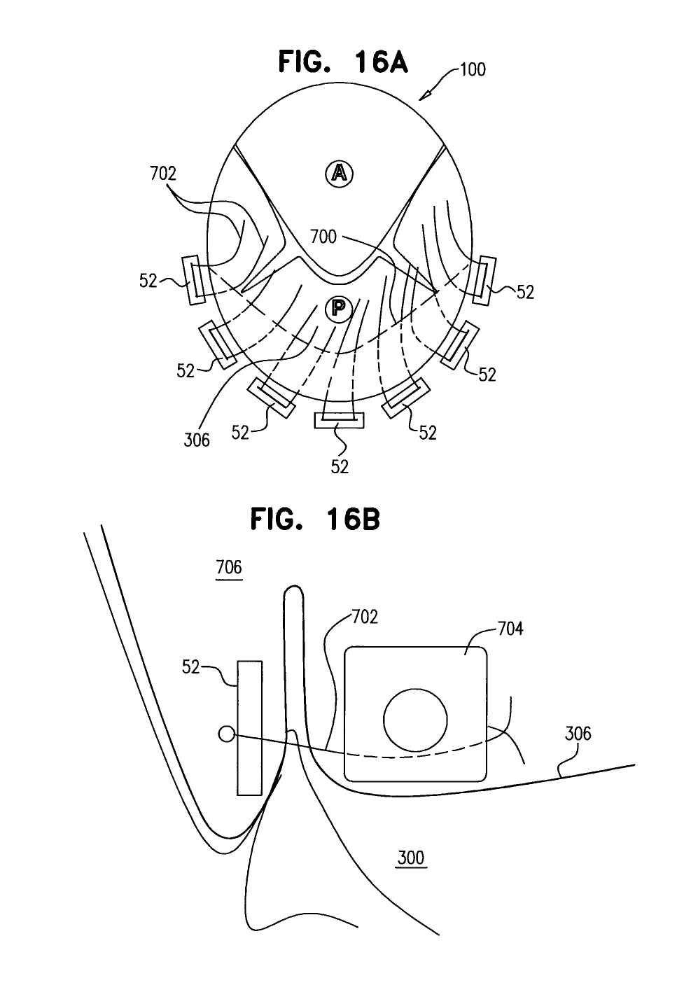

FIG. 2A shows the placement of five posterior sutures, using five respective pledgets 52. The position of the five posterior sutures of P2 is marked by the five pledgets 52. Everting pledgeted sutures may be employed to achieve a reduction in the height of P2, such as using the non-resection placation techniques described hereinbelow with reference to FIGS. 16A-B. When no reduction in P2 height is required, simple conventional sutures may be used. FIG. 2B additionally shows the placement of anterior sutures (for clarity of illustration, the three central posterior sutures shown in FIG. 2A are not shown in FIG. 2B). FIG. 2F shows an exemplary configuration of the prosthesis after sutures have been placed, as described below, but before the prosthesis has been coupled to the native valve. FIG. 2G shows an exemplary configuration of the prosthesis sutured down in place, after the sutures have been tightened and tied, placing the prosthesis in its final location. The prosthesis is typically used to reduce the pathologically dilated native annulus (typically at least in the anterior-posterior direction), in order to restore proper valve function.

Annuloplasty prosthesis 10 is typically implanted during an open heart surgical procedure. The surgeon places the prosthesis at mitral valve 100, such that:
posterior bar 30 is positioned along a middle scallop (P2) of a posterior leaflet of mitral valve 100;
first (lateral) side bar 36A is positioned along a lateral scallop (P1) of the posterior leaflet and extending to a lateral fibrous trigone 110A of the mitral valve; and
second (medial) side bar 36B is positioned along a medial scallop (P3) of the posterior leaflet and extending to a medial fibrous trigone 110B of the mitral valve.

The surgeon couples first and second articulating joints 48 and 50 of prosthesis 10 to the native junctions (small commissures) of P2, as follows:
first articulating joint 48 is coupled to the mitral valve at the junction of P1 and P2, and
second articulating joint 50 is coupled to the mitral valve at the junction of P2 and P3.
   Any movement of prosthesis 10 at these locations is generally dispersed in the commissural space, and therefore is unlikely to tear the leaflets.

The surgeon couples the joints of the prosthesis to the mitral valve using tissue anchoring elements, such as sutures (pledgeted or non-pledgeted). For some applications, as shown in FIGS. 2C-G, two primary sutures 120 (or other tissue-coupling elements) are used to couple first and second articulating joints 48 and 50, respectively, to two sides of P2 directly opposite the small commissures (minor clefts) at the junctions of P1 and P2, and P2 and P3, respectively. A P1/P2 primary suture 120 is passed through the fabric covering of the prosthesis (not shown) and first posterior side bar ring 42A of first side bar 36A, and then pledgeted to the mitral valve annulus using everting mattress suturing, preferably pledgeted, such as described hereinbelow with reference to FIGS. 16A-B. The suture then passes through first posterior bar ring 44 and the fabric covering. A P2/P3 primary suture 120 likewise couples second posterior side bar ring 42B and second posterior bar ring 46 to the mitral valve annulus. These pledgeted primary sutures opposite the P1/P2 and P2/P3 junctions thus anchor posterior bar 30 and the rings of the posterior and side bars down into their exact anatomical locations. If necessary to reduce the height of P2, one or more of several techniques may be used: a sliding plasty, a folding plasty, and/or a plication plasty. In a plication plasty, the pledgeted primary sutures and the other pledgeted posterior sutures plicate a portion of P2, such as shown in FIG. 16B, described hereinbelow, leaving the remainder of P2 with a desired height. Each of pledgets 52 may be 3×7 mm, for example. (For clarity of illustration, FIG. 2C shows only primary sutures 120 and the sutures at the trigones; additional sutures are also used, as shown in some of the other figures.)

For some applications, as shown in FIGS. 2C and 2E-G, the surgeon further couples first anterior end 38A of first side bar 36A to lateral fibrous trigone 110A, and second anterior end 38B of second side bar 36B to medial fibrous trigone 110B. The surgeon uses tissue coupling elements, such as sutures. For applications in which the anterior ends of the side bars are shaped so as to define anterior side bar rings 51A and 51B, as described hereinabove with reference to FIG. 1A, the surgeon typically sutures the rings to the trigones.

For applications in which fabric covering 20 is provided, the surgeon additionally couples the covering to the mitral valve, such as by suturing. Fabric covering 20 is typically used to couple the side bars and the poster bar to the mitral valve, at several points along each bar. Optionally, the surgeon may additionally suture anterior connecting segment 62 of fabric covering 22 (shown in FIGS. 1B and 2E-G) to the mitral valve, typically between about 1 o'clock and about 11 o'clock. Alternatively, the surgeon may cut off anterior connecting segment 62. For example, the total number of sutures, including those that also couple articulating joints 48 and 50 and/or anterior ends 38A and 38B, may be at least 8, such as between 9 and 16.

FIG. 2F shows an exemplary configuration of the prosthesis after sutures have been placed, but before the prosthesis has been coupled to the native valve. In this configuration, in addition to the two primary sutures 120 described above, three additional posterior pledgeted everting mattress sutures are used to couple posterior bar 30 to the native valve, such as using techniques described hereinbelow with reference to FIGS. 16A-B. The remaining anterior sutures are typically conventional, non-everting sutures. One or more of these anterior sutures optionally passes through first and second anterior side bar rings 51A and 51B (FIG. 1A), respectively. For some applications, the surgeon may deem it necessary to place the base of the posterior leaflet between the pledgets and the annuloplasty prosthesis, such as described hereinbelow with reference to FIGS. 16A-B.

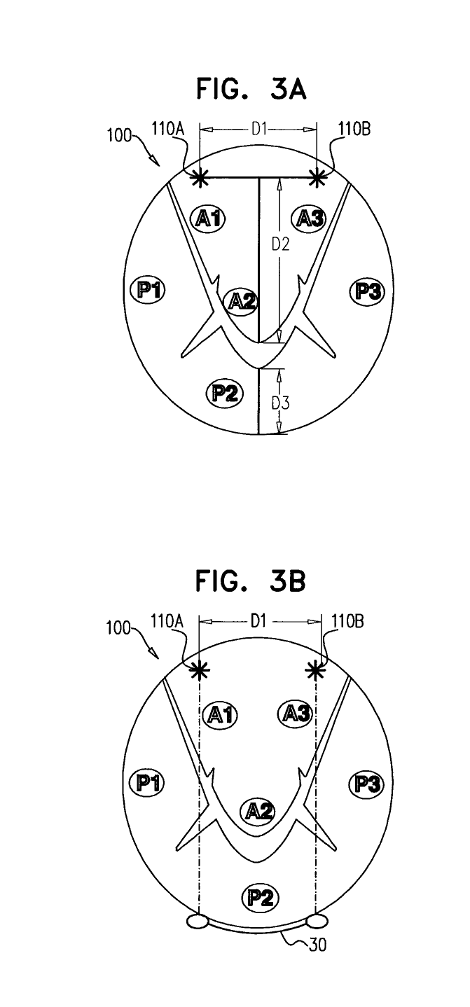

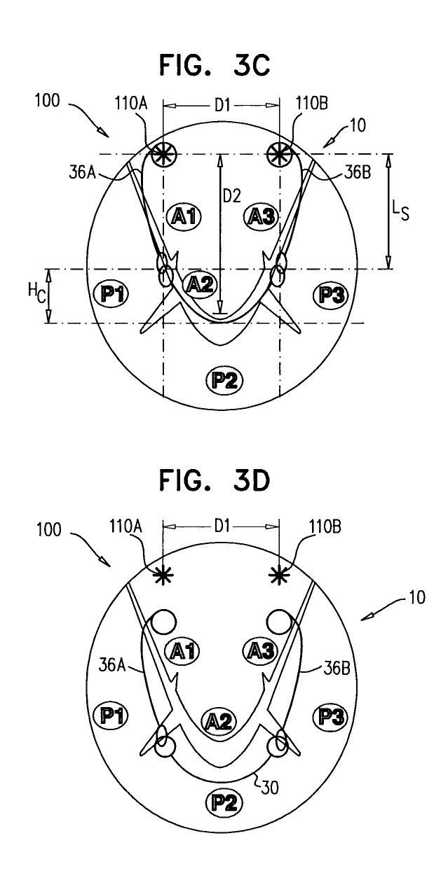

Reference is now made to FIGS. 3A-D, which are schematic illustrations of techniques for selecting bar lengths of the annuloplasty prosthesis responsively to anatomical measurements, in accordance with respective applications for the present invention. For some applications, a kit is provided that comprises a plurality of annuloplasty prostheses 10 respectively having posterior bars 30 of at least two different lengths, and side bars 36A and 36B of at least two different lengths (both side bars in any given prosthesis 10 typically have the same length, or the same length+/−20%). The kit typically includes prostheses having at least four different combinations of posterior length and side length, such as at least nine or at least 12 different combinations. Typically, the ratio of poster length to side length varies among at least some of the prostheses of the kit. Typically, at least two (e.g., at least three) of the prostheses of the kit have respective posterior bars having the same length, and respective first side bars having different lengths (and typically, respective second bars having the same lengths as the respective first side bars, or lengths +/−20% the lengths of the respective first side bars). Typically, at least two (e.g., at least three) of the prostheses of the kit have respective first side bars having the same length, and respective posterior bars having different lengths (and typically, respective second bars having the same lengths as the respective first side bars, or lengths +/−20% the lengths of the respective first side bars).

The variety of lengths and ratios in the kit allows the surgeon to select a prosthesis that is appropriate for the particular measurements of each particular patient's mitral valve, taking the length of the posterior bar and the length of the side bars into account separately. For some applications, different kits are provided having different size ranges and/or average sizes, such as a kit intended for generally smaller valves, and a kit intended for generally larger valves. For example, the exemplary kit described in Table 1 below has generally smaller sizes than the exemplary kit described in Table 2 below.

The following tables show the dimensions of the prostheses of two exemplary kits.

TABLE 1

| Prosthesis # | Length of posterior bar | Length of side bars |
|---|---|---|
| 1 | 25-27 mm (e.g., 26 mm) | 25-27 mm (e.g., 26 mm) |
| 2 | 25-27 mm (e.g., 26 mm) | 29-31 mm (e.g., 30 mm) |
| 3 | 28-31 mm (e.g., 30 mm) | 28-31 mm (e.g., 30 mm) |
| 4 | 28-31 mm (e.g., 30 mm) | 32-36 mm (e.g., 34 mm) |

More generally, the kit may comprise, for example, the four combinations resulting from two different lengths of the poster bar, and two different lengths of the side bars.

TABLE 2

| Prosthesis # | Length of posterior bar | Length of side bars |
|---|---|---|
| 1 | 36-40 mm (e.g., 38 mm) | 36-40 mm (e.g., 38 mm) |
| 2 | 36-40 mm (e.g., 38 mm) | 38-42 mm (e.g., 40 mm) |
| 3 | 36-40 mm (e.g., 38 mm) | 40-44 mm (e.g., 42 mm) |
| 4 | 41-44 mm (e.g., 42 mm) | 41-44 mm (e.g., 42 mm) |
| 5 | 41-44 mm (e.g., 42 mm) | 44-47 mm (e.g., 45 mm) |
| 6 | 41-44 mm (e.g., 42 mm) | 47-50 mm (e.g., 48 mm) |
| 7 | 45-48 mm (e.g., 46 mm) | 45-48 mm (e.g., 46 mm) |
| 8 | 45-48 mm (e.g., 46 mm) | 49-52 mm (e.g., 50 mm) |
| 9 | 45-48 mm (e.g., 46 mm) | 53-56 mm (e.g., 54 mm) |

More generally, the kit may comprise, for example, the nine combinations resulting from three different lengths of the poster bar (small, medium, and large), and three different lengths of the side bars (small, medium, and large).

(As mentioned above, for applications in which the bars are curved, the lengths given in Tables 1 and 2 are measured in straight lines between the ends of the bars, rather than along the curves.)

For some applications, in order to select the prosthesis from the kit having the most appropriate dimensions, the surgeon measures different first and second anatomical distances of the mitral valve, and selects the posterior bar length responsively to the first anatomical distance, and the side bar length responsively to the second anatomical distance.

For some applications, when the side bar length is selected responsively to the second anatomical distance, the side bar length is selected responsively to the second anatomical distance minus the height of curvature H_{C} of the posterior bar, as described hereinbelow with reference to FIG. 3C. For some applications, the side bar lengths labeled in the kit take this subtraction into account for the surgeon (i.e., reflect the second anatomical distance without the subtraction, even though the side bars themselves are sized based on the subtraction). The manufacturer thus provides the kit having prostheses comprising posterior bars and lateral bars that provide appropriate heights and widths for the measured anatomical distances. The surgeon selects the most appropriately-sized prosthesis from the kit based on the measured first and second anatomical distances. For other applications, the side bar lengths labeled in the kit reflect this subtraction (i.e., are net values after the subtraction); for these applications, the surgeon subtracts the posterior bar curvature height H_{C} from the measured second anatomical distance, and selects a prosthesis based on the net height.

Reference is again made to FIGS. 3A and 3B. For some applications, the first anatomical distance is an inter-trigone distance D₁ between lateral and medial fibrous trigones 110A and 110B. The surgeon selects one of the annuloplasty prostheses of the kit based on a comparison of the inter-trigone distance and the lengths of the posterior bars of the annuloplasty prostheses. Selecting a posterior bar having a length approximately equal to that of the inter-trigone distance helps restore the width of P2 to the inter-trigone distance. (The width of P2 equals the distance between (a) the junction of P1 and P2 and (b) the junction of P2 and P3.)

Reference is again made to FIGS. 3A and 3C. For some applications, the second anatomical distance is a height of the anterior leaflet (A) of the mitral valve, which is the distance D₂ between (a) the center of a line between lateral and medial fibrous trigones 110A and 110B and (b) the tip of the middle scallop (A2) of the anterior leaflet (A). Before measuring the height of the anterior leaflet, the surgeon typically stretches the posterior portion of the anterior leaflet in a posterior direction, such as by using a hook or other surgical tool. The surgeon selects one of the annuloplasty prostheses of the kit based on a comparison of the height D₂ and the lengths of side bars 36A and 36B of the annuloplasty prostheses. Typically, side bars are selected that have a length equal L_{S} to approximately that of height D₂ (such as between 90% and 110% of D₂), typically minus the posterior bar curvature height H_{C}, as shown in FIG. 3C. The posterior bar curvature height H_{C} is measured at the center of the posterior bar in the anterior-posterior direction, and thus extends between the center of the posterior bar and the center of a hypothetical line that connects the ends of the posterior bar.

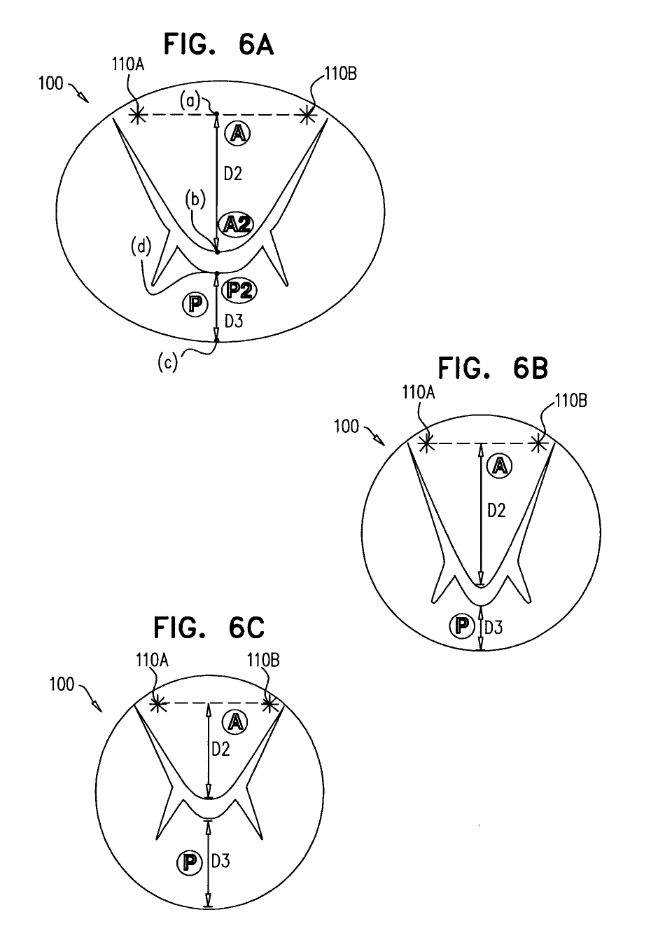

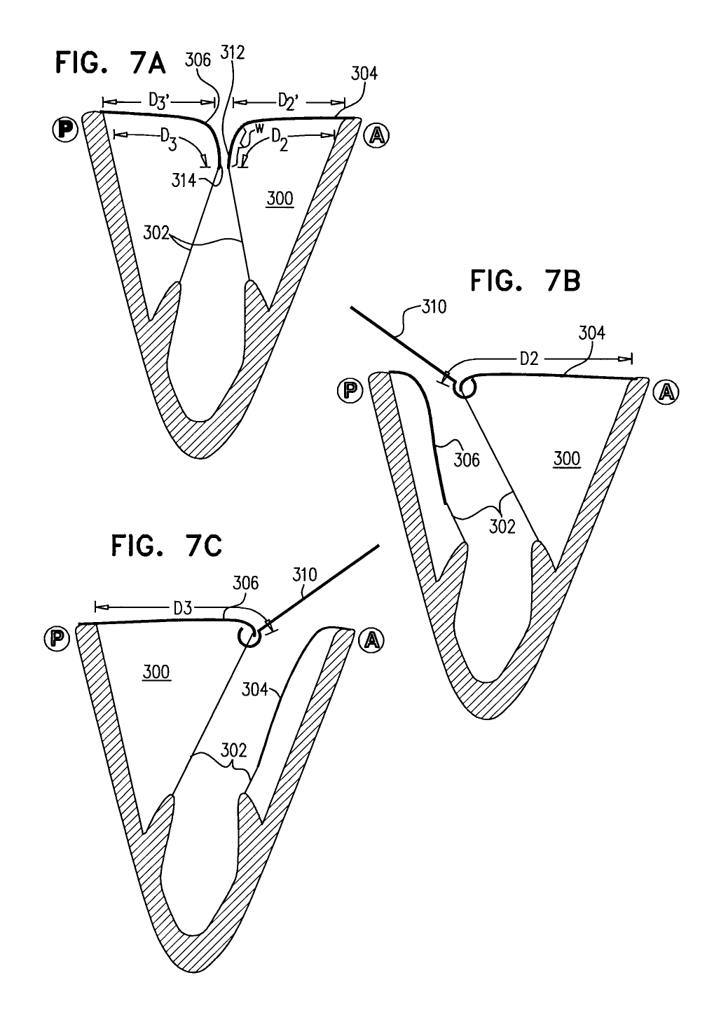

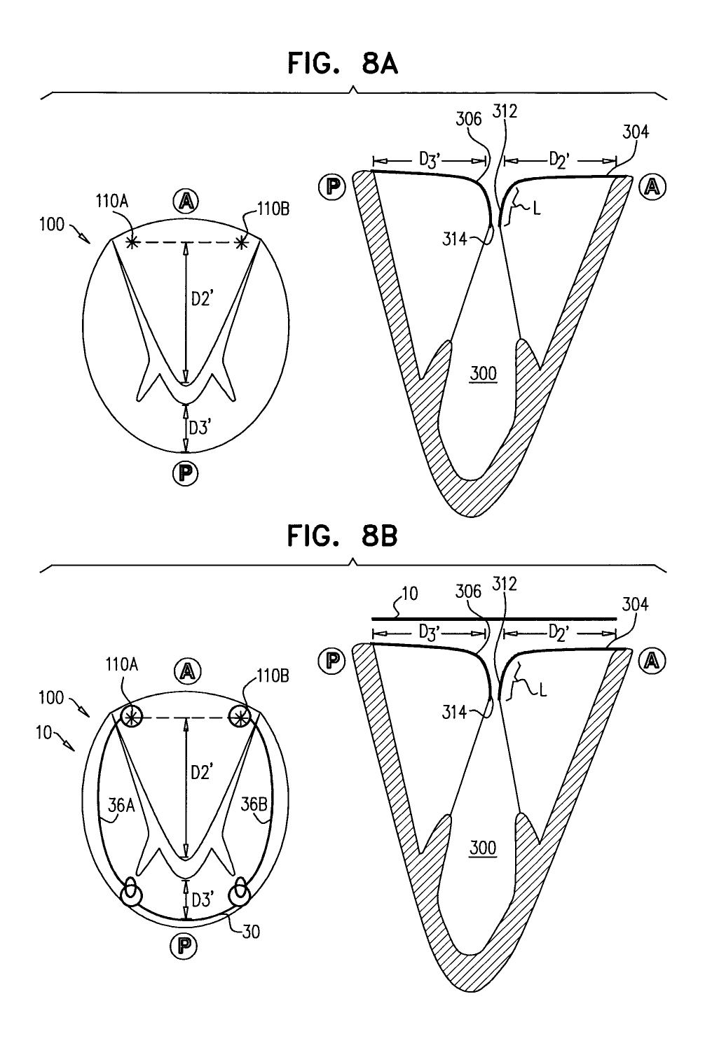

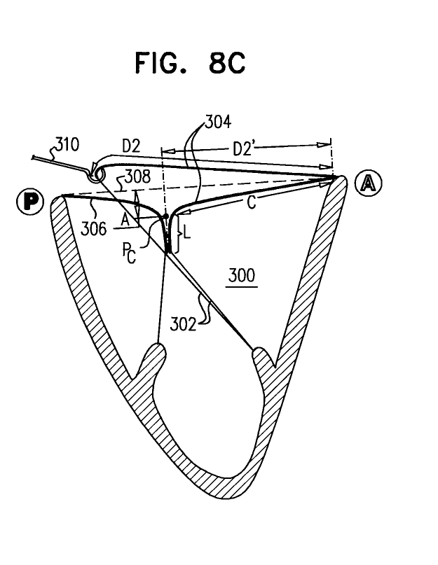

Alternatively, for some applications, as described hereinbelow with reference to FIGS. 6A-8B, longer side bars are selected responsively to the height D₂ and the height D₃ of the posterior leaflet (e.g., the sum of D₂ and D₃ less a value indicative of twice a coaptation length, as described hereinbelow with reference to FIGS. 8A-B and Equation 4, and typically minus the posterior bar curvature height H_{C}, as described hereinabove with reference to FIG. 3C, and, optionally, minus an additional constant to account for the depth of coaptation in the beating heart, as described hereinbelow with reference to FIG. 8C). The anterior-poster height of the prosthesis equals the length L_{S} of the side bars plus the posterior bar curvature height. (Distances D₂ and D₃ are shown schematically in the views of FIGS. 3A and 3C; during a surgical procedure only portions of D₂ and D₃ are actually visible from the atrium, because portions of the leaflets are drawn down into ventricle by chordae tendineae, as described hereinbelow with reference to FIGS. 7A-C.) (D₃ is thus a third anatomical distance.)

For some applications, when the side bar length L_{S} is selected responsively to both D₂ and D₃, the side bar length L_{S} is selected responsively to D₂ and D₃ minus the height of curvature H_{C} of the posterior bar, as described hereinabove with reference to FIG. 3C. For some applications, the side bar lengths L_{S} labeled in the kit take this subtraction into account for the surgeon (i.e., reflect the sum of D₂ and D₃ less the value indicative of twice the coaptation length, without the subtraction of posterior bar curvature, even though the side bars themselves are sized based on the subtraction). The manufacturer thus provides the kit having prostheses comprising posterior bars and lateral bars that provide appropriate heights and widths for the measured anatomical distances. The surgeon selects the most appropriately-sized prosthesis from the kit based on the measured inter-trigone distance, D₂, and D₃. For other applications, the side bar lengths L_{S} labeled in the kit reflect this subtraction (i.e., are net value after the subtraction); for these applications, the surgeon subtracts the posterior bar curvature height from the measured D₂ and D₃. Alternatively or additionally, for some applications, the side bars labeled in the kit take into account the subtraction of the value indicative of twice the coaptation length (i.e., reflect the sum of D₂ and D₃ without the subtraction of the indicative value, even though the side bars themselves are sized based on the subtraction of the indicative value).

For some applications, a length L_{S} of the longest one of the posterior bars in the kit is between 180% and 220% of a length L_{S} of the shortest one of the posterior bars in the kit.

Table 3 sets forth the exemplary lengths L_{S} of the posterior bars and side bars set forth in Table 2, and, in addition, the ratios between these lengths.

TABLE 3

| Prosthesis | Length of | Length of | Ratio of side bars to |
|---|---|---|---|
| # | posterior bar | side bars | posterior bar |
| 1 | 38 mm | 38 mm | 1.00 |
| 2 | 38 mm | 40 mm | 1.05 |
| 3 | 38 mm | 42 mm | 1.11 |
| 4 | 42 mm | 42 mm | 1.00 |
| 5 | 42 mm | 45 mm | 1.07 |
| 6 | 42 mm | 48 mm | 1.14 |
| 7 | 46 mm | 46 mm | 1.00 |
| 8 | 46 mm | 50 mm | 1.09 |
| 9 | 46 mm | 54 mm | 1.17 |

Typically, at least two of the prostheses of the kit have ratios of side bar to posterior bar lengths that differ from each other. For example, at least three of the prostheses may have different ratios. These varying ratios, and not merely sizes, enable the kit to accommodates native valves having varying sizes and proportions.

Reference is again made to FIG. 3D, which shows the skeleton of prosthesis 10 superimposed on dilated native mitral valve 100. (The figure is for illustrative purposes only to show the measurements of the prosthesis 10 with reference to the measured anatomical distance. In actual use, the surgeon typically does not place the prosthesis on the valve until after placing the sutures, as described hereinabove with reference to FIGS. 2A-G.) The lengths of posterior bar 30 and side bars 36A and 36B have been selected according to the specific measurements of this valve, as described hereinabove with reference to FIGS. 3A-C. As can be seen, prosthesis 10 is smaller than the native valve.

Reference is made to FIG. 4, which is a schematic illustration of potential movements of bars of annuloplasty prosthesis 10, in accordance with an application of the present invention. Because posterior bar 30 is free to articulate with side bars 36A and 36B, the posterior bar is free to rotate upward into the atrium, in response to natural leaflet tensions, as schematically indicated by an arrow 150 (the posterior bar is also free to move downward). Such upward rotation may reduce tension on P2. Similarly, anterior connecting segment 62 of fabric covering 22 is free to rotate upward into the atrium, as illustrated by an arrow 152. Alternatively or additionally, side bars 36A and 36B may rotate downward toward the ventricle, as schematically indicated by arrows 154 (the side bars are also free to move upward, outward, and inward, but not in the anterior-posterior direction). (It is noted that posterior and side bars are rigid, and do not change their shapes as they rotate.) The bars of the prosthesis thus naturally adopt the position of least leaflet tensions. This rotation of the bars and the anterior connecting segment may cause the prosthesis to assume a saddle shape, imitating the three-dimensional saddle shape of the mitral annulus, thereby reducing systolic strain, as reported in the above-mentioned article by Padala M et al.

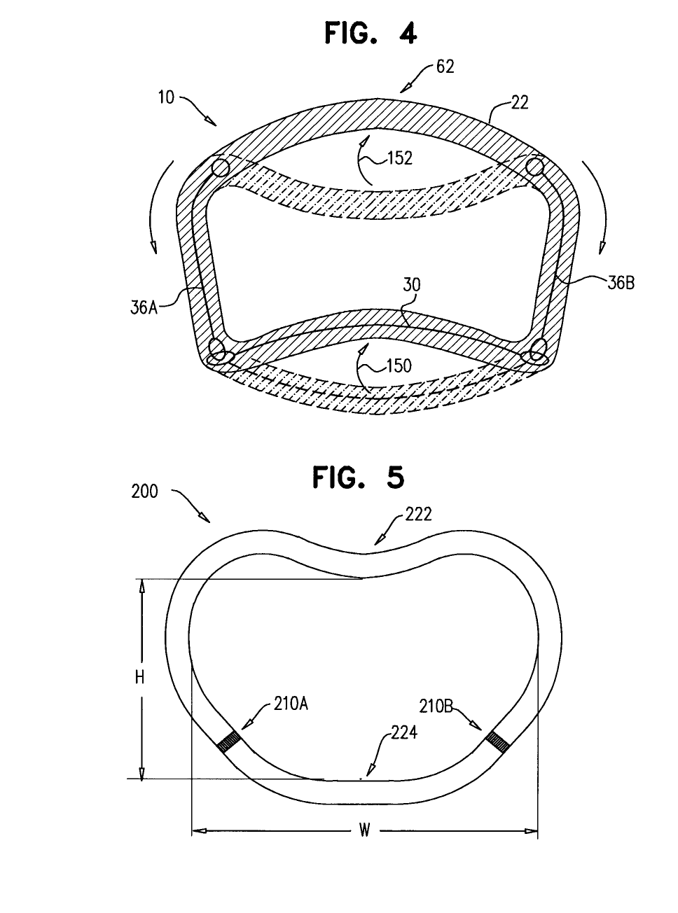

Reference is made to FIG. 5, which is a schematic illustration of an annuloplasty prosthesis 200, in accordance with an application of the present invention. Annuloplasty prosthesis 200 may be rigid, semi-rigid, flexible, or partially rigid and partially flexible, may comprises a ring or a band, and may implement techniques described in the references mentioned in the Background of the Application hereinabove. The annuloplasty prosthesis may be a complete prosthesis, as shown in the figure, or a band (i.e., partial prosthesis), as is known in the art (configuration not shown).

For some applications, annuloplasty prosthesis 200 is coupled to the native mitral valve by suturing the prosthesis at first and second locations 210A and 210B thereon to two sides of P2 directly opposite the small commissures (minor clefts) at the junctions of P1 and P2, and P2 and P3, respectively. For some applications, a P1/P2 primary suture is passed through prosthesis 200 at location 210A and then pledgeted to the mitral valve annulus using everting mattress suturing, such as described hereinbelow with reference to FIGS. 16A-B (in addition, see FIG. 2C above for a schematic illustration of the anatomical positioning of the pledgets). The suture then passes through prosthesis 200 again at location 210A, near the location at which the suture initially passed through the prosthesis. A P2/P3 primary suture likewise couples the prosthesis at location 210B to the mitral valve annulus. These pledgeted primary sutures opposite the P1/P2 and P2/P3 junctions thus anchor locations 210A and 210B of prosthesis 200 down into their exact anatomical locations. Additional posterior and anterior sutures are typically also applied, such as described hereinabove with reference to FIGS. 2A-B and 2E-F, or as known in the art. Each of pledgets 52 may be 3×7 mm, for example.

For some applications, annuloplasty prosthesis 200 has at least two visually-sensible fiducial designators 220A and 220B at locations 210A and 210B, respectively, in order to indicate to the surgeon the proper locations for applying the primary sutures, as described above. (The prosthesis may have additional fiducial designators at other locations, such as is known in the annuloplasty art.) Fiducial designator 220A is typically placed at between 7 and 8 o'clock (e.g., at about 7.5 o'clock), and fiducial designator 220B is typically placed at between 4 and 5 o'clock (e.g., at about 4.5 o'clock), assuming that 12 o'clock is at a center 222 of the anterior portion of prosthesis 200 (if this portion is provided), and 6 o'clock is at a center 224 of the posterior portion of the prosthesis. (Equivalently, fiducial designator 220A is typically placed at between 210 and 240 degrees (e.g., at about 225 degrees), and fiducial designator 220B is typically placed at between 120 and 150 degrees (e.g., at about 135 degrees), assuming that 0 degrees is at anterior center 222 (if this portion is provided), and 180 degrees is at posterior center 224.) For other applications, annuloplasty prosthesis 200 does not have fiducial designators 220A and 220B.

For some applications, a kit is provided that comprises a plurality of annuloplasty prostheses 200 having different dimensions. The surgeon measures inter-trigone distance D₁ between lateral and medial fibrous trigones 110A and 110B, as shown FIG. 3A, and selects the kit based on a comparison of the measured inter-trigone distance and a distance between fiducial designators 220A and 220B. Typically, the surgeon selects the prosthesis having the distance between fiducial designators 220A and 220B that is closest to the measured inter-trigone distance, such as +/−10% of the inter-trigone distance. Typically, the surgeon selects the anterior-posterior diameter of the prosthesis as in a complete ring (the sum of the anterior leaflet height and the posterior leaflet height, minus twice the coaptation length), or according to the anterior leaflet height only, in which case the posterior leaflet height is reduced to twice the coaptation length.

For some applications, a kit is provided that comprises a plurality of conventional annuloplasty prostheses, and/or annuloplasty prostheses 200, having different dimensions (the prostheses may or may not have fiducial designators 220A and 220B). For some applications, in order to select the prosthesis from the kit having the most appropriate dimensions, the surgeon measures different first and second anatomical distances of the mitral valve, and selects a first dimension of the prosthesis responsively to the first anatomical distance, and a second dimension of the prosthesis responsively to the second anatomical distance. For some applications, the first anatomical distance is an inter-trigone distance D₁ between lateral and medial fibrous trigones 110A and 110B, as shown FIG. 3A. For some applications, the second anatomical distance is a height of the anterior leaflet (A) of the mitral valve, which is the distance D₂ between (a) the center of a line between lateral and medial fibrous trigones 110A and 110B and (b) the tip of the middle scallop (A2) of the anterior leaflet (A), as shown in FIG. 3A. Alternatively, for some applications, the second anatomical distance is the height D₂ of the anterior leaflet and the height D₃ of the posterior leaflet, as described hereinbelow with reference to FIGS. 6A-8B. For example, the second dimension of the prosthesis may be selected responsively to the sum of D₂ and D₃ less a value indicative of twice a coaptation length, as described hereinbelow with reference to FIGS. 8A-B and Equation 4.

For some applications, at least two (e.g., at least three) of the prostheses in the kit have the same side-to-side width W and different respective anterior-posterior heights H (dimensions labeled in FIG. 5). For some applications, the kit includes at least two (e.g., at least three) sets of prostheses, each of which sets includes at least two (e.g., at least three) prostheses having the same width W and different heights H.

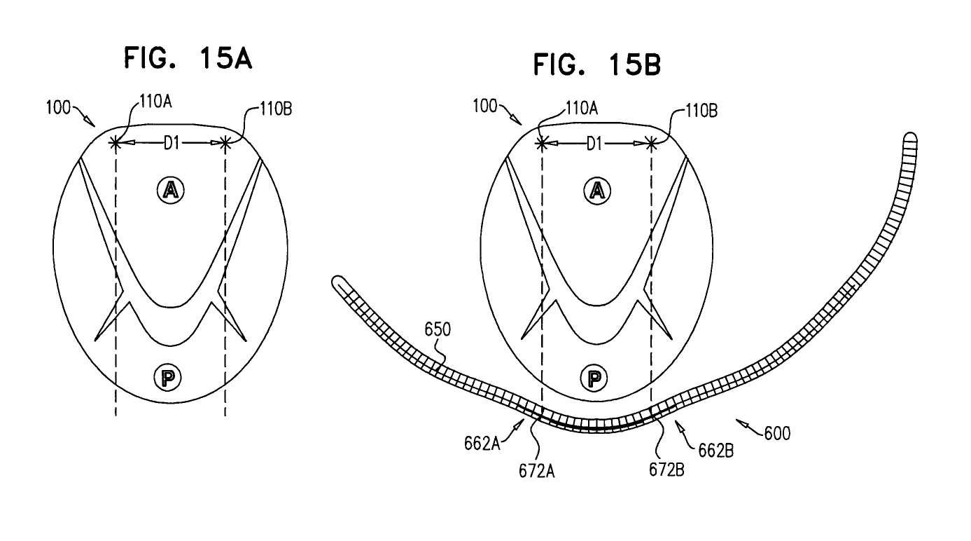

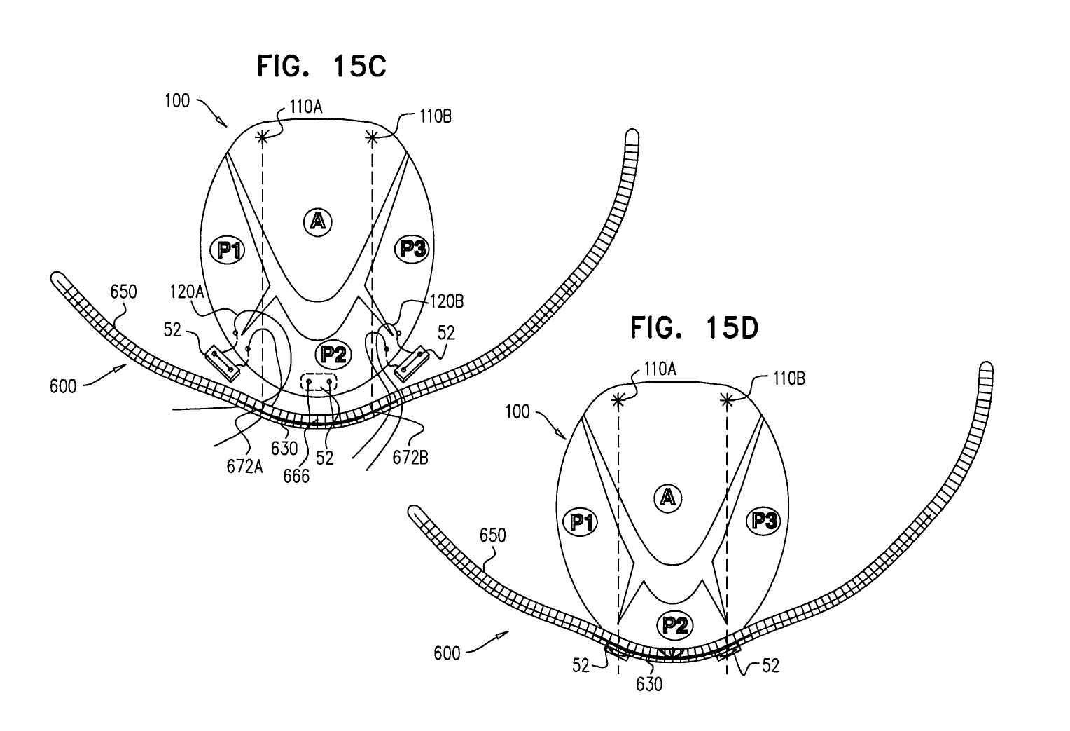

Reference is now made to FIGS. 6A-C, 7A-C, and 8A-B, which are schematic illustrations of a technique for sizing and implanting an annuloplasty prosthesis at a native mitral valve, in accordance with an application of the present invention. Although FIG. 8B shows the technique used to implant annuloplasty prosthesis 10, described hereinabove with reference to FIGS. 1A-B, the technique may also be used to implant annuloplasty prosthesis 600, described hereinbelow with reference to FIGS. 14A-B and 15A-G, and other annuloplasty prostheses, such as conventional annuloplasty prostheses, or annuloplasty prosthesis 200, described hereinabove with reference to FIG. 5.

Conventional open-heart annuloplasty procedures are typically single-leaflet repair procedures, which restore function only to the anterior leaflet. The annuloplasty prosthesis is sized to support the anterior leaflet, and the height of the posterior leaflet is reduced by plicating or resecting a portion of the posterior leaflet to remove any portion of the posterior leaflet considered redundant (typically to a height of two times the coaptation length of the anterior and posterior leaflets). After the procedure, the posterior leaflet serves mainly to provide a coaptation countersurface for the functional anterior leaflet.

While this conventional procedure often produces acceptable results, it has at least two disadvantage. First, this conventional procedure sometimes causes SAM, as discussed hereinabove in the Background of the Application. Second, this conventional procedure generally produces relatively small valve orifice areas. Post-procedure orifice areas of between two and three cm2 are common, compared to orifice areas of about 6 cm2 in a healthy valve.

The resulting orifice area depends in part on the relative heights of the posterior and anterior leaflets, which vary among patients. As shown in FIGS. 6A-C (and FIGS. 3A and 3C, as described hereinabove), the height of the anterior leaflet (A) is the distance D₂ between (a) the center of a line between lateral and medial fibrous trigones 110A and 110B and (b) the tip of the middle scallop (A2) of the anterior leaflet (A). The height of the posterior leaflet (P) is the distance D₃ between (c) the mitral valve annulus at the middle of P2 (6 o'clock) and (d) the tip of the middle scallop (P2) of the posterior leaflet (P). (In FIGS. 6A-C, the leaflets are shown stretched during the implantation procedure.)

In most patients, the anterior leaflet height is considerably greater than the posterior leaflet height, such as at least 1.5 times greater, such as shown in FIG. 6A. In other patients with anterior leaflet dominance, the difference is even greater; for example, the anterior leaflet height may be about 2.5 times the posterior leaflet height, such as shown in FIG. 6B. In both of these groups of patients, the conventional procedure typically produces an adequate orifice area. However, in some patients with posterior leaflet dominance, the posterior leaflet height is about equal to the anterior leaflet height, such as shown in FIG. 6C. In these latter patients, the conventional procedure generally produces an undesirably small orifice area, and indeed relatively large posterior leaflet heights are associated with a higher incidence of SAM.

In contrast, the technique of this application of the present invention restores function to both the anterior and posterior leaflets. The annuloplasty prosthesis is sized based on the combined heights of both the anterior and posterior leaflets, and the height of the posterior leaflet is typically not reduced. This sizing prevents undersized prostheses, when the anterior leaflet is relatively small. As a result, the technique does not produce the relatively small orifice areas common in conventional procedures. In addition, the technique provides space in the ventricle for the native valve to expand and positions the valve away from the LV outflow tract, thereby reducing the risk of SAM. This technique is particularly appropriate for patients with posterior leaflet dominance, and may also be appropriate for patients with any ratio of anterior leaflet height to posterior leaflet height.

FIGS. 7A-C show schematic side-views of the leaflets and a left ventricle 300, in accordance with an application of the present invention. The figures also show two exemplary chordae tendineae 302. In order to measure the height D₂ of anterior leaflet 304, the surgeon pulls the posterior portion of anterior leaflet 304 in a posterior direction, such as by using a hook 310 or other surgical tool, as shown in FIG. 7B. This stretching and straightening is necessary to obtain an accurate measurement, because a portion of the anterior leaflet is drawn down into ventricle 300 by chordae tendineae 302, in order to provide a coaptation surface 312 with a similar coaptation surface 314 near the tip of the posterior leaflet. Coaptation surfaces 312 and 314 typically share a common coaptation length L. In the figure, D₂′ represents the portion of anterior leaflet 304 that remains facing the atrium, and does not provide coaptation surface 312.

Similarly, in order to measure the height D₃ of posterior leaflet 306, the surgeon pulls the anterior portion of posterior leaflet 306 in an anterior direction, such as by using hook 310 or other surgical tool, as shown in FIG. 7C. This stretching and straightening is necessary to obtain an accurate measurement, because a portion of the posterior leaflet is drawn down into ventricle 300 by chordae tendineae 302, in order to provide coaptation surface 314. In the figure, D₃′ represents the portion of posterior leaflet 306 that remains facing the atrium, and does not provide coaptation surface 314.

The heights of the anterior and posterior leaflets can thus be expressed using the following equations:

D₂=D₂′+L (Equation 1)

D₃=D₃′+L (Equation 2)

Reference is made to FIGS. 8A and 8B, which are schematic top- and side-views of the leaflets and left ventricle 300, in accordance with an application of the present invention. FIG. 8A shows only the anatomy, while FIG. 8B further shows a simplified schematic illustration of annuloplasty prosthesis 10 in place at the valve. (It is noted that in the left figures of FIGS. 8A and 8B the leaflets are not stretched by the surgeon.) An appropriately-sized annuloplasty prosthesis is selected based on a comparison of the anterior-poster height of the prosthesis with the sum of anterior leaflet height D₂ and posterior leaflet height D₃. In the case of full-sized prosthesis 10, as shown in FIGS. 8A-B, the anterior-poster height of the prosthesis equals the length of side bars 36A and 36B plus the curvature height H_{C} of posterior bar 30, as described hereinabove with reference to FIG. 3C. The length of posterior bar 30 is typically selected as described hereinabove with reference to FIGS. 3A-C. (For clarity of illustration, fabric covering 20 of prosthesis 10 is not shown in FIG. 8A, although the fabric covering is typically provided, as shown in FIG. 1B.) As mentioned above, the technique may also be used to implant other annuloplasty prostheses, such as conventional annuloplasty prostheses, or annuloplasty prosthesis 200, described hereinabove with reference to FIG. 5. For some applications, a kit is provided that comprises a plurality of conventional annuloplasty prostheses, and/or annuloplasty prostheses 200, having different dimensions (the prostheses may or may not have fiducial designators 220A and 220B), as described hereinabove with reference to FIG. 5. An appropriately-sized prosthesis is selected from the kit based at least in part on the measured anterior and posterior heights, as described below.

The anterior-posterior height H of the prosthesis is selected based on the combined height of the portions of anterior and posterior leaflets 304 and 306 that face the atrium, as shown in FIG. 8A, as expressed by the following equation:

H=D₂′+D₃′ (Equation 3)

However, as described above, D₂′ and D₃′ typically are not in practice directly measured.

Instead, D₂ and D₃ are measured. In order for the prosthesis to enable the natural leaflets to provide coaptation surfaces 312 and 314, twice coaptation length L is subtracted from the sum of D₂ and D₃, as expressed by the following equation:

H=D₂+D₃−2L (Equation 4)

Because the coaptation length L does not vary substantially between patients, and is typically between 8 and 12 mm, a constant can be used to estimate 2L. The constant typically has a value between 1.2 and 2.5 cm, such as 2 cm. Alternatively, for some applications, the coaptation length L is measured or estimated for the individual patient, and used in Equation 4.

For some applications in which posterior bar 30 is curved, the appropriate side bar length L_{SB} is selected by subtracting the posterior bar curvature height H_{PBC} from the anterior-posterior height H of the prosthesis, as expressed by the following equation:

L_{SB}=H−H_{PBC}=D₂+D₃−2L−H_{PBC} (Equation 5)

Alternatively (and mathematically equivalently), the appropriately-sized side bar is selected based on a comparison of D₂+D₃−2L and L_{SB}+H_{PBC}. For example, H_{PBC} may be between 2 and 4 mm, such as 3 mm.

(Equation 5 is general, and the common single leaflet repair may be considered a special case of the equation. In other words, in the particular case in which D₃=2L, i.e., the posterior leaflet height equals twice the coaptation length L, D₃ and 2L cancel each other, and L_{SB}=H−H_{PBC}, as described hereinabove for the applications in which the posterior leaflet height is not measured.)

Reference is now made to FIG. 8C, which shows a schematic side-view of the leaflets and left ventricle 300, in accordance with an application of the present invention. FIG. 8C shows anterior leaflet 304 in two positions: (1) pulled in a posterior direction during a surgical procedure, such as by using hook 310, and (2) relaxed partially drawn into ventricle 300, during beating of the heart.

Optionally, when performing the calculations described above, an additional coaptation-depth correction constant C_{CDC} is subtracted from the anterior-posterior height H to account for the depth of coaptation in the beating heart. As shown in FIG. 8C, in a beating heart, a coaptation point P_{C} between the leaflets is a depth A below a plane 308 defined by the annulus of the mitral valve. Equations 1-5, described hereinabove, are most accurate when D₂′ is measured in plane 308. However, the measurement technique described above in practice calculates a distance C along anterior leaflet 304 (equal to measured D₂ less the coaptation length L). Because the leaflet slants downward somewhat towards coaptation point P_{C}, the calculated value of D₂′ is slightly inflated. To correct for this inflation, the additional coaptation-depth correction constant C_{CDC} may be subtracted from the calculated value of D₂′. Similarly, the additional coaptation-depth correction constant C_{CDC} may be subtracted from the calculated value of D₁′. The constant C_{CDC} is typically between 1.5 and 2 mm, so that a combined constant 2C_{CDC} for D₁′+D₂′ is typically between 3 and 4 mm. The constant C_{CDC} may, for example, be estimated by assuming that D₂′ is actually 30 mm, and depth A is 10 mm, in which case distance C is about 31.6 mm (according to the Pythagorean theorem).

Thus, optionally, for greater precision, Equation 4 may be restated as follows:

H=D₂+D₃2L−2C_{CDC} (Equation 6)

Because the coaptation length L does not vary substantially between patients, and is typically between 8 and 12 mm, a constant can be used to estimate 2L. The constant for 2L typically has a value between 1.2 and 2.5 cm, such as 2 cm. As mentioned above, the combined constant 2C_{CDC} may be estimated to have a value of between 3 and 4 mm.

Similarly, for some applications in which posterior bar 30 is curved, for greater precision Equation 5 may be restated as follows:

L_{SB}=H−H_{PBC}=D₂+D₃−2L−2C_{CDC}−H_{PBC} (Equation 7)

This technique thus provides a full-sized prosthesis, which preserves the full orifice area of the native mitral valve, while maintaining the proper coaptation length L, in order to provide full closure of the valve.

For some applications, a kit is provided that includes prostheses 10 having different combinations of posterior and side bar lengths, as described hereinabove with reference to Tables 1 and 2. Typically, for use with the full-sized prosthesis technique, the lengths of the side bars are larger than those given in Tables 1 and 2. For example, the side bar lengths may be between 10% and 30% larger than those given in the tables.

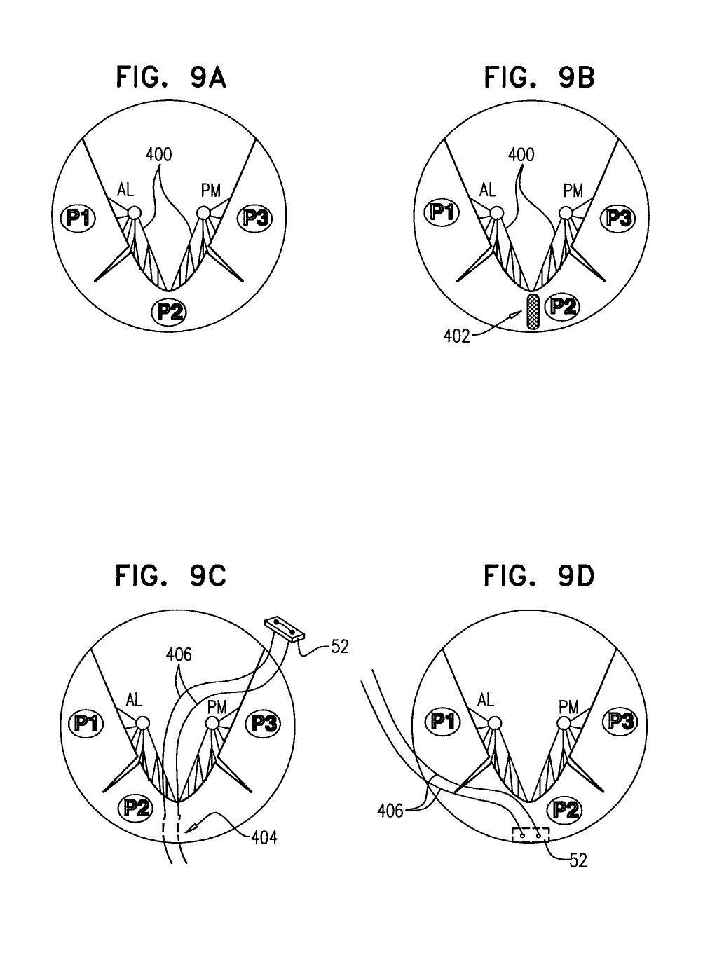

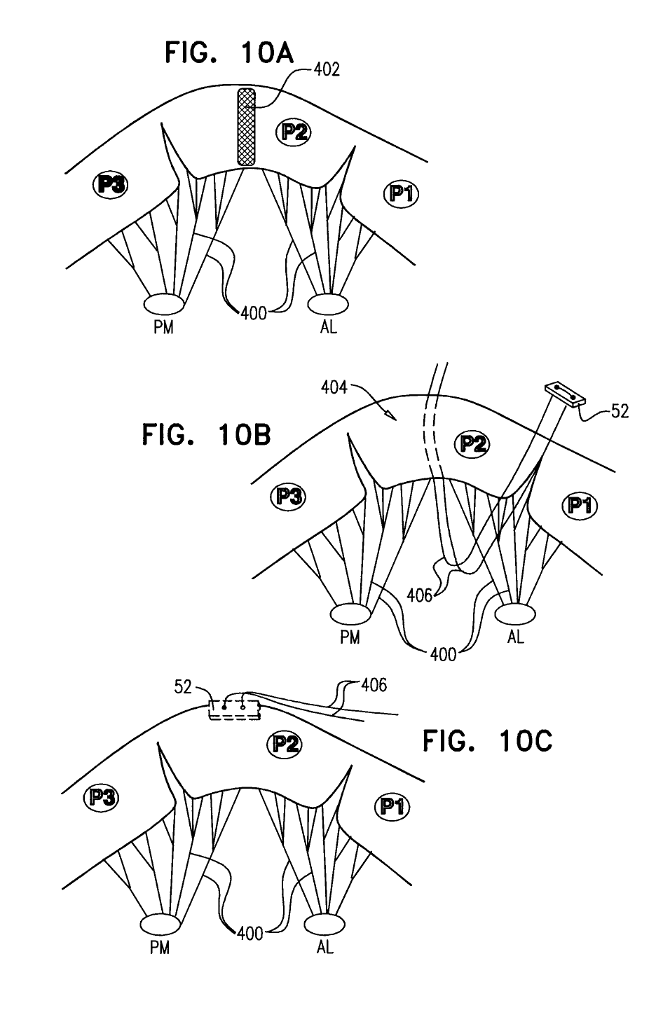

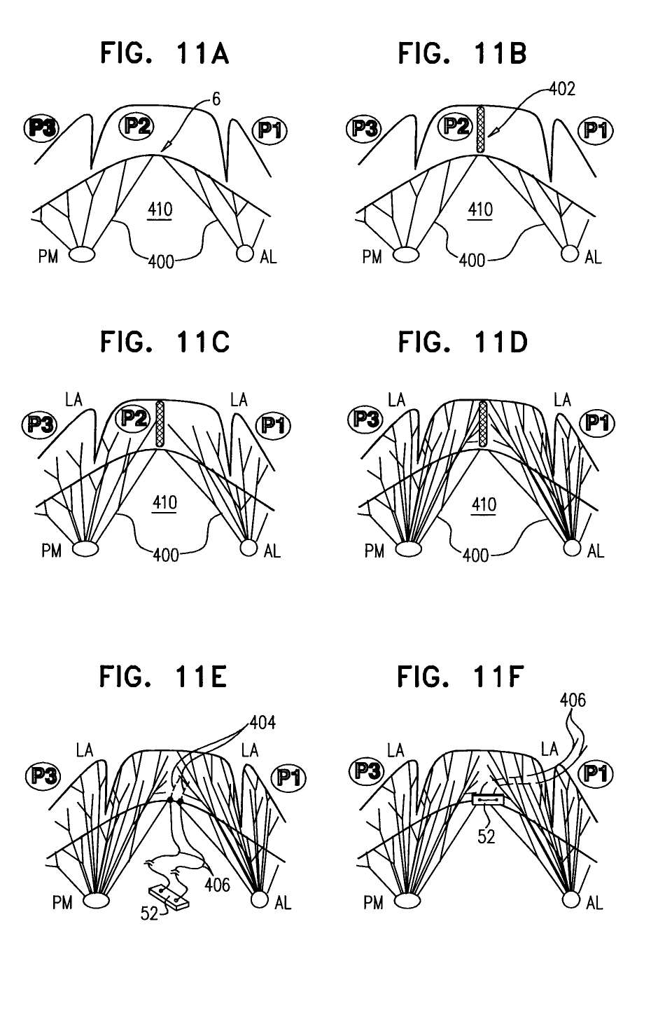

Reference is now made to FIGS. 9A-D, 10A-C, and 11A-F, which schematically illustrate a method for pledgeting an annuloplasty prosthesis to a mitral valve, in accordance with an application of the present invention. FIGS. 9A-D are views of the procedure from above (from the atrium), FIGS. 10A-C are views from anterior side, and FIGS. 11A-F are views from below (from the left ventricle). In FIGS. 9A-D, 10A-C, and 11A-F, the anterior leaflet is omitted for the sake of clarity.

In this application, a pledget is placed against the ventricular side of the mitral annulus on the border of the leaflet and the annulus, such that one or more sutures that pass through the pledget and the posterior leaflet at the mitral annulus and extend into the atrium (i.e., both ends of each of the one or more sutures extend into the atrium). The sutures couple the pledget to the ventricular side, and are used to couple an annuloplasty prosthesis to the posterior leaflet at the middle of the posterior mitral annulus at P2. While this pledgeting technique can be used in general for various mitral valve treatment procedures, it may be particularly appropriate for patients with anterior leaflet dominance, as described hereinabove with reference to FIG. 6B, who have posterior leaflets with a particularly small height.

A pledget 52 (such as a single pledget 52) is used below P2 at 6 o'clock (a center of a posterior portion of native valve) to provide strong anchoring of the prosthesis at the weakest portion of the mitral annulus, to which the fibrous skeleton does not extend. The lack of chordae tendineae at 6 o'clock substantially reduces the risk that the surgeon might inadvertently capture one or more of the chordae tendineae with the suture of the pledget, causing undesirable accordion pleating of the posterior leaflet at P2. The inventor believes that cardiac surgeons are generally unaware of this chordae tendineae-free region, which is surrounded by a dense array of chordae tendineae, as perhaps best seen in FIGS. 11D-F, discussed below. This pledgeting technique is typically, but not necessarily, performed during an open heart surgical procedure, as described with reference to FIGS. 9A-D, 10A-C, and 11A-F.

It is of particular importance to enforce the posterior annulus of the mitral valve at 6 o'clock (the annulus's nadir, labeled as “6” in FIG. 11A), because of the minimal support naturally provided at this location. 6 o'clock is the weakest location in the native valve, because there is no fibrous annulus at this location (the native mitral valve does not have a complete fibrous annulus). As a result, the mitral valve dilates in this posterior direction, as the posterior segment of the annulus bellows posteriorly. Conventional sutures sometimes do not adequately grasp this location. This optional technique of the present invention thus uses a sub-leaflet pledget at this location (because the leaflet tissue is strong), instead of conventional sutures at the unsupported annulus, for better coupling.

This technique may also help minimize the risk of dehiscence.

FIGS. 9A, 10A, and 11A show the posterior leaflet, an anterolateral (AL) papillary muscle, a posteromedial (PM) papillary muscle, and two groups of chordae tendineae 400, extending from these two papillary muscles, respectively.

FIGS. 9B, 10A, and 11B show a chordae tendineae-free region 402 at 6 o'clock. This region is between the regions of the two groups of chordae tendineae.

FIGS. 11A and 11B show the tertiary chordae, which are inserted into the mitral valve annulus. FIG. 11C additionally shows the secondary chordae, which are inserted into the leaflet itself, between the annulus and the free margin. FIG. 11D additionally shows the primary chordae, which are inserted into the free margin of the leaflets. (In FIGS. 11A-F, a left-ventricular posterior wall 410 can be seen.)

FIGS. 9C, 10B, and 11E show the passing of two needles 404 and a suture 406 through chordae tendineae-free region 402. Suture 406 has typically been coupled to a single pledget 52 before the sutures are passed through the tissue.

FIGS. 9D, 10C, and 11F show the posterior leaflet with pledget 52 in place below the leaflet at 6 o'clock.

At this stage of the procedure, the two ends of suture 406 are in the left atrium, at the middle of P2. The suture is used to provide a strong anchor for an annuloplasty prosthesis.

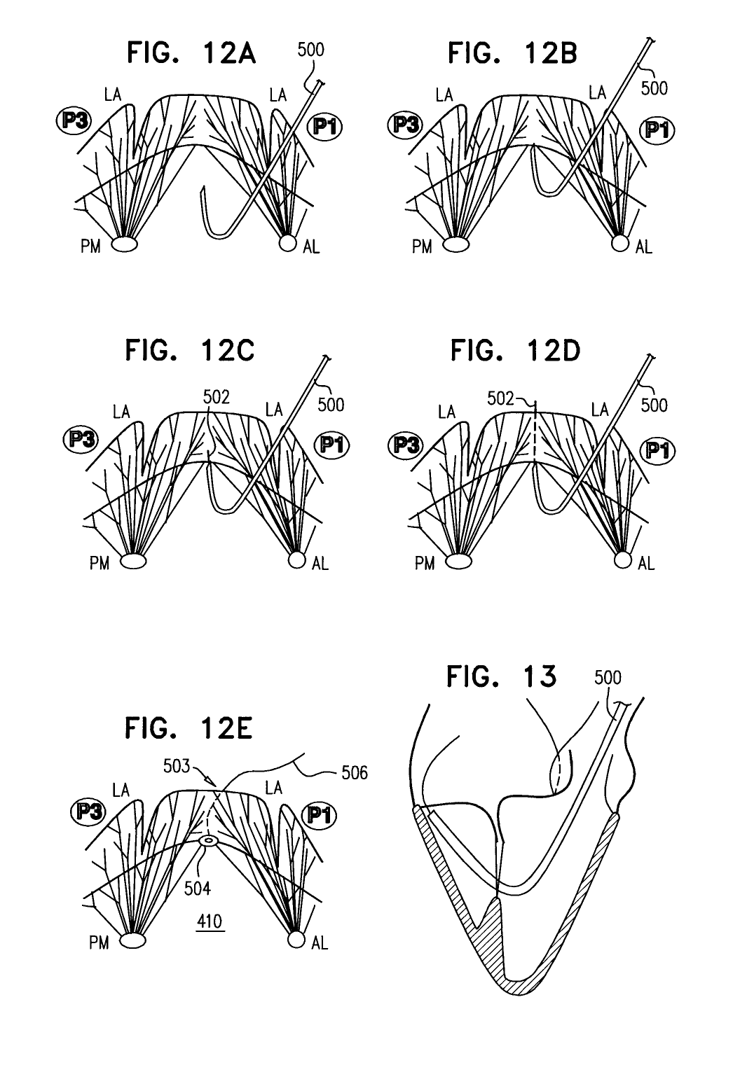

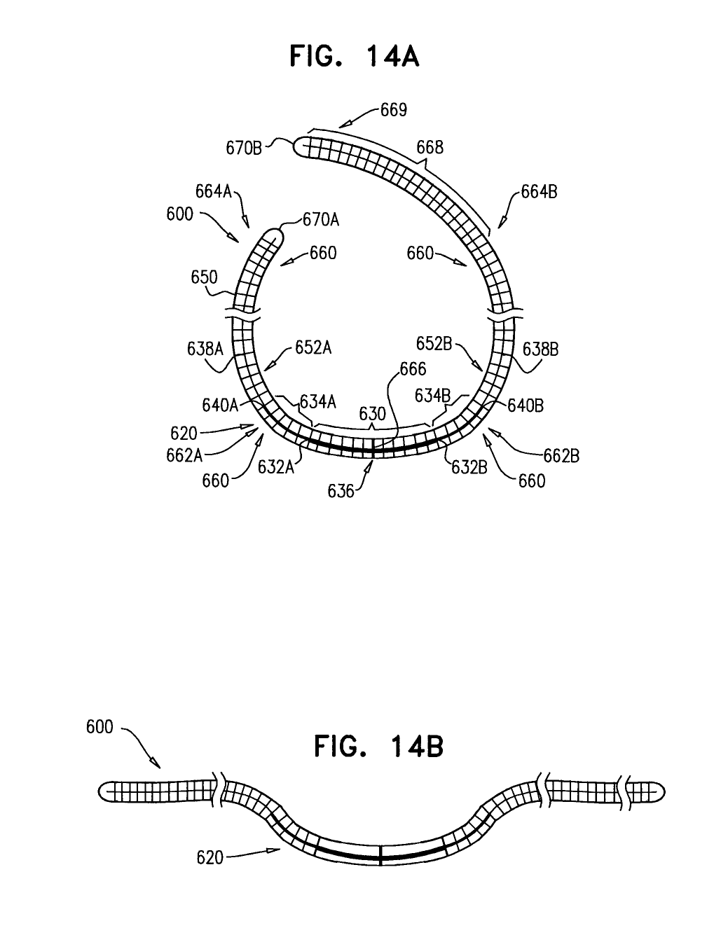

Reference is made to FIGS. 12A-E and 13, which schematically illustrate a transvascular method for coupling an annuloplasty prosthesis to a mitral valve, using an anchor 503, in accordance with an application of the present invention. FIGS. 12A-E are views of the procedure from below (from the left ventricle), in which the anterior leaflet is omitted for the sake of clarity. FIG. 13 is a lateral view. This procedure is performed using a delivery tool, which typically includes a catheter 500 and one or more guidewires and/or other wires, which may be introduced transvascularly (e.g., percutaneously) into the left ventricle via the aorta, as is known in the transvascular cardiac procedure art, such as over a guidewire, as is known in the art. The delivery tool is used to introduce and implant anchor 504, to which an annuloplasty prosthesis is subsequently coupled (the annuloplasty prosthesis is not shown in the figures).

As shown in FIG. 12A, catheter 500 is aimed at the base of the middle of the posterior leaflet, such as described hereinabove with reference to FIGS. 9B, 10A, and 11B. The catheter is typically positioned using echocardiogram and/or fluoroscopic guidance, and may be advanced over a guidewire (not shown), as is known in the art.

FIG. 12B shows the catheter properly positioned, just below the ring, at 6 o'clock (see the explanation provided above with reference to FIGS. 9B, 10A, and 11B for the significance of this location).

Typically, a sharp wire 502 (an element of the delivery tool) is used during the implantation procedure to penetrate the leaflet. FIG. 12C shows sharp wire 502 during initial penetration of the leaflet. FIG. 12D shows sharp wire 502 extending into the left atrial cavity after penetrating the leaflet.

FIG. 12E shows anchor 503, which comprises a ventricular anchor base 504 (e.g., comprising an umbrella anchor), and, fixed thereto, at least one elongated atrial anchoring member 506, which may comprise a wire and/or a hook, for example. For some applications, anchoring member 506 comprises a hollow wire (e.g., comprising a metal) that is shaped so define a channel therethrough. Anchoring member 506 is passed through the posterior leaflet (or, alternatively, through the mitral annulus) via sharp wire 502, such as by passing the anchoring member over sharp wire 502. (Alternatively, sharp wire 502 may first be withdrawn and replaced with another wire, as is known in the catheter art, and the anchoring member may be passed using the replacement wire.) The sharp wire (or replacement wire) is subsequently removed, along with the catheter, leaving anchoring member 506 extending into the atrium, and anchor base 504 positioned on and anchored to the ventricular side of the posterior leaflet of the mitral annulus, and anchoring member 506 extending to the atrium for use in coupling an annuloplasty prosthesis thereto. Anchor 503 is thus used instead of pledget 52, described hereinabove with reference to FIGS. 9B, 10A, and 11B.

For applications in which the anchor base comprises an umbrella anchor, the umbrella expands once in place (e.g., using an expandable stent), in order to provide anchoring to the annulus. Anchoring member 506 extends into the left atrial cavity, and the portion that extends into the atrium serves as a suture or hook for anchoring a annuloplasty prosthesis to the middle posterior annulus. For some applications, a conventional umbrella is used, as is known in the cardiac surgical art, albeit for non-anchoring purposes. For some applications, a plurality of anchors 503 are used. For some applications, anchoring member 506 comprises metal. For some applications, an elongated element other than a wire is used.

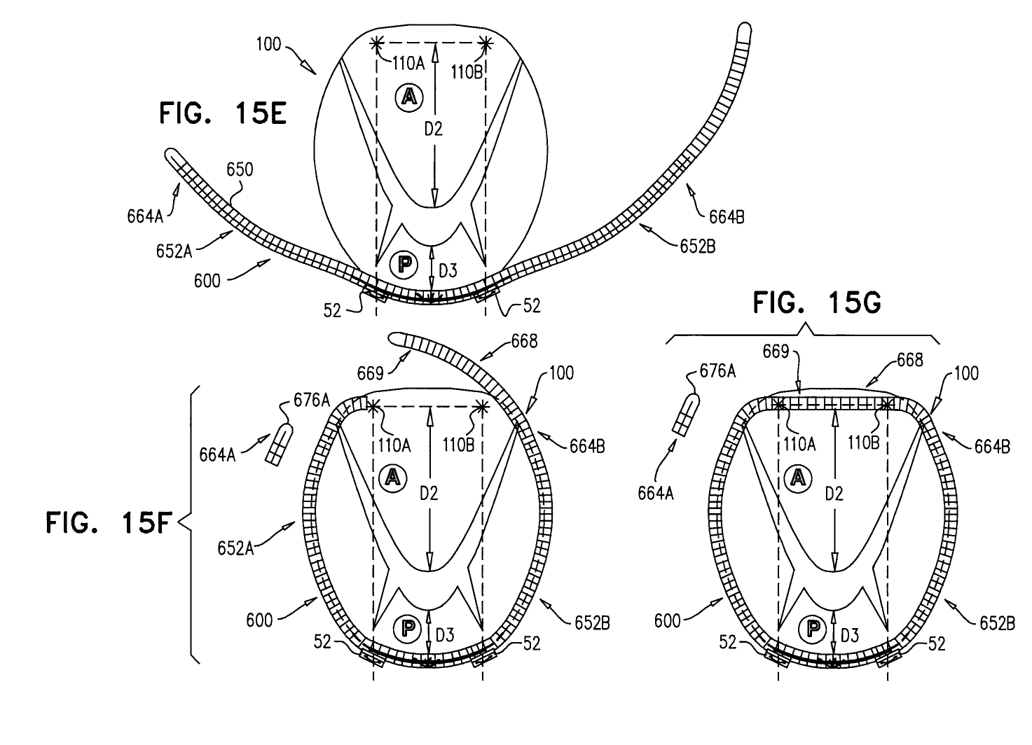

FIGS. 14A and 14B are schematic illustrations of an annuloplasty prosthesis 600, in accordance with an application of the present invention. FIG. 14A shows the prosthesis arranged approximately in a shape appropriate for implantation at the mitral valve, while FIG. 14B shows the prosthesis partially straightened. Prosthesis 600 is configured to be placed at a native mitral valve, such as described hereinbelow with reference to FIGS. 15A-G.

Prosthesis 600 comprises a fabric sleeve 622 and a skeleton 620 that is positioned within a portion of the fabric sleeve. For some applications, skeleton 620 consists entirely of metal, such as nitinium or titanium, or a metal alloy. Alternatively, all or a portion of the skeleton comprises a non-metal material, such as a synthetic resin or a plastic.

Skeleton 620 comprises:
a rigid posterior bar segment 630, which has first and second bar ends 632A and 632B;
optionally, first and second semi-rigid posterior segments 634A and 634B, which begin at first and second bar ends 632A and 632B, respectively, and extend away from a longitudinal center 636 of rigid posterior bar segment 630; and
optionally, first and second flexible side segments 638A and 638B, which begin at first and second anterior ends 640A and 640B of first and second semi-rigid posterior segments 634A and 634B, respectively, and extend away from longitudinal center 636.

As used in the present application, including in the claims, “rigid” means stiff and resisting bending; “flexible” means capable of bending easily without breaking; and “semi-rigid” means partly or moderately rigid, i.e., more rigid than “flexible” and less rigid than “rigid.”

Typically, rigid posterior bar segment 630 has a length of between 24 and 48 mm, such as between 24 and 34 mm, e.g., between 25 and 30 mm, such as between 25 and 27 mm, e.g., 26 mm. For some applications, rigid bar segment 630 is curved; for these applications, the length of the bar is measured in a straight line between the ends of the bar, rather than along the curve of the bar. Rigid posterior bar segment 630 typically comprises a metal, such as titanium, or a metal alloy.

For applications in which skeleton 620 comprises first and second semi-rigid posterior segments 634A and 634B, typically each the first and second semi-rigid posterior segments 634A and 634B has a length of at least 4 mm, no more than 15 mm, and/or between 4 and 15 mm, such as at least 4 mm, no more than 10 mm, and/or between 4 and 10 mm. For some applications in which skeleton 620 comprises first and second semi-rigid posterior segments 634A and 634B, the skeleton comprises a semi-rigid structural element, having (a) first and second portions that respectively define first and second semi-rigid posterior segments 634A and 634B, and (b) a third portion that longitudinally coincides with rigid posterior bar segment 630. For some applications, rigid posterior bar segment 630 passes through the portion of the semi-rigid structural element; for example, the semi-rigid structural element may comprise a metal stent, e.g., an elastic net-tube. For other applications, the portion of the semi-rigid structural element passes through rigid posterior bar segment 630; for example, the rigid posterior bar may comprise a metal tube. Similarly, for applications in which skeleton 620 further comprises first and second flexible side segments 638A and 638B, the flexible side segments may comprise separate structural elements coupled to anterior ends 640A and 640B.

Prosthesis 600 further comprises a sleeve 650, which typically functions as a sewing ring for coupling prosthesis 600 to the native mitral valve. Sleeve 650:
comprises a flexible and substantially non-extensible fabric material, i.e., it substantially cannot stretch or longitudinally contract. For example, the fabric may comprise Dacron® or Teflon®;
covers rigid posterior bar segment 630, and, typically, covers the entire skeleton 620, and optionally extends beyond the ends of the skeleton; and
includes a first portion 652A that extends at least 20 mm beyond (i.e., in an interior direction away from longitudinal center 636 of rigid posterior bar segment 630) first bar end 632A and a second portion 652B that extends at least 20 mm beyond second bar end 632B (these distances are measured along the sleeve, even if the sleeve is not straight).

Because of this design, annuloplasty prosthesis 600 is nearly rigid in the anterior-posterior direction, and highly flexible in the side-to-side direction. In particular, first and second anterior ends 670A and 670B of sleeve 650 are free to move in the side-to-side direction, as well as up and down (to provide flexibility in three dimensions), as first and second portions 652A and 652B of the sleeve articulate with rigid posterior bar segment 630. Rigid posterior bar segment 630 has a relatively low burden, and has some freedom of motion. This freedom of motion provides the rigid posterior bar segment with some shock absorption capability, and diffuses the bending movement, thus causing the rigid posterior bar segment to be break-resistant.

For some applications, a total length of sleeve 650 (before trimming) is at least 100 mm, no more than 180 mm, and/or between 100 and 180 mm, such as at least 100 mm, no more than 150 mm, and/or between 100 and 150 mm. For some applications, a length of first portion 652A (from longitudinal center 636 to first anterior end 670A, prior to any trimming, as describe below) is between 60 and 80 mm, such as 70 mm. For some applications, a length of second portion 652B (from longitudinal center 636 to second anterior end 670B, prior to any trimming, as describe below) is: (a) between 70 and 90 mm, such as 70 mm, for applications in which the prosthesis is configured to be implanted as a band, i.e., partial prosthesis; or (b) between 110 and 130 mm, such as 110 mm, for application in which the prosthesis is configured to be implanted as a complete prosthesis. For some applications, a length of sleeve 650 beyond first bar end 632A is at least 20 mm, no more than 70 mm, and/or between 20 and 70 mm (such as at least 25 mm, no more than 50 mm, and/or between 25 and 50 mm), and a length of sleeve 650 beyond second bar end 632B is at least 20 mm, no more than 100 mm, and/or between 20 and 100 mm (such as at least 25 mm, no more than 90 mm, and/or as between 25 and 90 mm). For some applications, a length of the sleeve beyond second bar end 632B is at least 20 mm greater than (e.g., between 25 and 44 mm greater than) a length of the sleeve beyond first bar end 632A, in order to provide an inter-trigone portion 668, for coupling to the native mitral valve as described hereinbelow with reference to FIG. 15G. Thus, for these applications, the sleeve is asymmetrical about longitudinal center 636. For other applications in which the sleeve is deployed as an open prosthesis, and thus does not include inter-trigone portion 668, the sleeve is typically symmetrical about longitudinal center 636. (The lengths described in this paragraph are measured along the sleeve, even if the sleeve is not straight.)

Sleeve 650 has a plurality of visually-sensible fiducial designators 660, i.e., indicators, at respective longitudinal locations, typically distributed (typically equally spaced) along at least 95% (e.g., 100%) of sleeve 650 (typically to the ends or near (e.g., within 5 mm of) the ends of the sleeve). Alternatively, fiducial designators 660 are distributed only along a posterior portion of the sleeve, such as on both longitudinal sides of longitudinal center 636 up to no more than 40 mm from the longitudinal center. For some applications, the fiducial designators may be spaced at one- or two-millimeter intervals. The fiducial markers thus function similar to a ruler.

Fiducial designators 660 typically are arranged to indicate longitudinal center 636 of rigid posterior bar segment 630. For example, designators 600 may include a central posterior fiducial designator 666 located at longitudinal center 636; alternatively, for example, two of designators may be located on opposite sides of longitudinal center 636 near the longitudinal center, thereby indicating the longitudinal center. The central posterior fiducial designator is typically distinguished from the other fiducial designators (for example, it may be slightly darker or wider, or of a different color from, the other fiducial designators). Thus, fiducial designators 600 (other than central posterior fiducial designator 666, if provided) are useful for measuring the distance from longitudinal center 636 toward first and second anterior ends 670A and 670B of sleeve 650.

For some applications, fiducial designators 660 comprise a material woven into the sleeve. Alternatively or additionally, for some applications, fiducial designators 660 comprise ink. For some applications, fiducial designators 660 are shaped as respective lines that surround at least half of a perimeter of 650 sleeve (i.e., a perimeter in cross-section) at respective longitudinal locations. For some applications, the fiducial designators are periodically distinguished (e.g., are darker or wider, or of a different color) along the sleeve, such as one in every five or one in every ten designators, such as on a conventional ruler. For example, the designators may be have a first level of difference every 5 mm, and a second, greater level of difference every centimeter, such as on a conventional ruler.

For some applications, a plurality (e.g., at least 3, 4, or 5) of fiducial designators 660 serve as first posterior fiducial designators 662A located longitudinally between (a) first bar end 632A and (b) 15 mm beyond first bar end 632A, inclusive, and a plurality (e.g., at least 3, 4, or 5) of fiducial designators 660 serve as second posterior fiducial designators 662B located longitudinally between (a) second bar end 632B and (b) 15 mm beyond second bar end 632B, inclusive. (These fiducial designators are typically not visibly distinguished from the other fiducial designators.) Pairs of first posterior fiducial designators 662A and second posterior fiducial designators 662B are spaced apart at distances representative of typical inter-trigone distances. These distances may be used for identifying two locations along the sleeve for coupling to the posterior annulus along the P2 leaflet of the native mitral valve, as described hereinbelow with reference to FIGS. 15C-D. For example, a first pair consisting of a first one of first posterior fiducial designators 662A and a first one of second posterior fiducial designators 662B may be spaced apart by 26 mm, and a second pair consisting of a second one of first posterior fiducial designators 662A and a second one of second posterior fiducial designators 662B may be spaced apart by 28 mm. Additional pairs may be spaced apart at distances including one or more of 30 mm, 32 mm, 34 mm, and 36 mm, for example. More generally, the distances may be whole numbers, such as even whole numbers, typically having a range having a lower end of between 25 mm and 28 mm, such as 26 mm, and an upper end of between 32 and 46 mm, such as 36 mm. As mentioned above, one of first posterior fiducial designators 662A is typically located longitudinally at first bar end 632A, and one of second posterior fiducial designators 662B is typically located longitudinally at second bar end 632B; these fiducial designators may comprise the first pair of fiducial designators mentioned above. Optionally, the distances between the pairs are indicated on the sleeve; alternatively, the distances are set forth in accompanying instructions.

For some applications, a plurality of fiducial designators 660 serve as first anterior fiducial designators 664A and a plurality of fiducial designators 660 serve as second anterior fiducial designators 664B. Pairs of first anterior fiducial designators 664A and second anterior fiducial designators 664B may be used for indicating where to trim the sleeve to achieve a desired anterior-posterior length, as described hereinbelow with reference to FIGS. 15E-F. First and second anterior fiducial designators 664A and 664B are typically located at least 40 mm beyond first and second bar ends 632A and 632B, respectively.

Typically, prosthesis 600 is rigid along rigid posterior bar segment 630, and is flexible or semi-rigid along at least 75% (e.g., at least 90% or 100%) of first portion 652A of sleeve 650 and along at least 75% (e.g., at least 90% or 100%) of second portion 652B of sleeve 650. For some applications, prosthesis 600 is flexible or semi-rigid (a) between first bar end 632A and a posterior-most one of first anterior fiducial designators 664A and (b) between second bar end 634B and a posterior-most one of second anterior fiducial designators 664B. For some applications, prosthesis 600 is flexible or semi-rigid at all longitudinal locations other than along rigid posterior bar segment 630. For some applications, prosthesis 600 is flexible or semi-rigid for at least 46 mm beyond first bar end 632A and at least 46 mm beyond second bar end 632B, such as at least 90 mm beyond second bar end 632B. For some applications, prosthesis 600 is flexible at all locations (a) beyond 15 mm beyond first bar end 632A and (b) beyond 15 mm beyond second bar end 632B.

For some applications, prosthesis 600 further comprises a plurality of tissue anchoring elements, such as sutures or other tissue coupling elements, e.g., helical coupling elements or tissue screws.

Reference is now made to FIGS. 15A-G, which are schematic illustrations of a method for implanting annuloplasty prosthesis 600, in accordance with an application of the present invention. For some applications, annuloplasty prosthesis 600 is provided in a single size, which, when deployed as described below, accommodates a wide range of native mitral valve dimensions. Alternatively, for other applications, annuloplasty prosthesis 600 is provided in a small number of sizes, such as two or three, each of which, when deployed as described below, accommodates a range of native mitral valve dimensions.

Typically, annuloplasty prosthesis 600 is implanted at native mitral valve 100 such that prosthesis 600 restores a length of the middle scallop (P2) annulus of a posterior leaflet of the mitral valve to the inter-trigone distance. Annuloplasty prosthesis 600 is typically implanted during an open heart surgical procedure.

As shown in FIG. 15A, the surgeon measures inter-trigone distance D₁ between lateral and medial fibrous trigones 110A and 110B of native mitral valve 100.

As shown in FIG. 15B, the surgeon selects two locations 672A and 672B on sleeve 650 responsively to measured inter-trigone distance D₁. For some application, the surgeon selects a pair of fiducial designators 660 consisting of one of first posterior fiducial designators 662A and one of second posterior fiducial designators 662B, which have a distance therebetween selected responsively to inter-trigone distance D₁. The surgeon typically selects the pair of fiducial designators having a distance therebetween closest to inter-trigone distance D₁ (such that a distance between location 672A and longitudinal center 636 of rigid posterior bar segment 630 equals half of measured inter-trigone distance D₁, and a distance between location 672B and longitudinal center 636 of rigid posterior bar segment 630 equals half of measured inter-trigone distance D₁). Alternatively, the surgeon may selects a pair of fiducial designators having a distance therebetween equal to between 90% and 110% of inter-trigone distance D₁.

As shown in FIGS. 15C-D, rigid posterior bar segment 630 is positioned along a middle scallop (P2) of the posterior leaflet (P) of mitral valve 100. Typically, but not necessarily, as shown in FIG. 15C, sleeve 650 is coupled at longitudinal center 636 of rigid posterior bar segment 630 to the annulus of posterior leaflet at the middle of the posterior mitral annulus at P2 (at 6 o'clock). As mentioned above, longitudinal center 636 is indicated by fiducial designators 660, such as by central posterior fiducial designator 666, if provided. For some applications, this coupling comprises suturing, and, optionally, pledgeting, which may be performed using the techniques described hereinabove with reference to FIGS. 9A-D, 10A-C, and 11A-F.

The surgeon then couples locations 672A and 672B of sleeve 650 to the annulus of the posterior leaflet of the native mitral valve, using tissue anchoring elements, such as sutures (pledgeted or non-pledgeted). Typically, as shown in FIGS. 15C-D, two primary sutures 120A and 120B (or other tissue-coupling elements) are used to couple locations 672A and 672B of sleeve 650, respectively, to two sides of P2 directly opposite the small commissures (minor clefts) at the junctions of P1 and P2, and P2 and P3, respectively. A P1/P2 primary suture 120A is passed through the annulus at the junction of P1 and P2, then through location 672A of sleeve 650. Suture 120A is optionally coupled to the mitral valve annulus using everting mattress suturing, preferably pledgeted, such as described hereinbelow with reference to FIGS. 16A-B. A P2/P3 primary suture 120B likewise couples location 672B of sleeve 650 to the mitral valve annulus. These pledgeted primary sutures opposite the P1/P2 and P2/P3 junctions thus anchor rigid posterior bar segment 630 down into its exact anatomical location. If necessary to reduce the height of P2, one or more of several techniques may be used: a sliding plasty, a folding plasty, and/or a plication plasty. In a plication plasty, the pledgeted primary sutures and the other pledgeted posterior sutures plicate a portion of P2, such as shown in FIG. 16B, described hereinbelow, leaving the remainder of P2 with a desired height. Each of pledgets 52 may be 3×7 mm, for example.

For applications in which skeleton 620 comprises first and second semi-rigid posterior segments 634A and 634B, as described hereinabove with reference to FIGS. 14A-B, and locations 672A and 672B are along these semi-rigid posterior segments, rigid posterior bar segment 630 provides complete rigidity along most of the base of the posterior leaflet, while the semi-rigid posterior segments provide some rigidity along the remainder of the base of the posterior leaflet. This configuration enables a unisized prosthesis to be effectively used on native valves having different sizes, by providing the benefit of full rigidity to a sufficiently long portion (such as 26 mm) of the posterior leaflet annulus to prevent bellowing of the leaflet, and providing some support to the remainder of the posterior leaflet annulus. For some applications of the present invention, one goal is to provide a single type of device, which will be amenable to fit all native valve sizes and proportions, while maintaining the basic principles of providing P2 with rigid support, the size of the inter-trigone distance of the particular patient, using straightforward surgical steps. In order to provide rigid support to the weakest point of the posterior valve annulus, the center of the rigid posterior bar is typically absolutely rigid, fit for the smallest size native valve, i.e., typically 25-26 mm. Typically, on both ends the rigid posterior bar continues with semi-rigid segments, typically having a length of 4-10 mm on each side. The sum of the length of the rigid posterior bar and the semi-rigid segments provides up to 46 mm of posterior rigid support. This design allows a manufacturer to provide the annuloplasty prosthesis is a single size (“unisize” or one-size-fits-all). Nevertheless, the manufacturer may prefer to provide the annuloplasty prosthesis in sets including two or three sizes, for example for inter-trigone distances of 25-30 mm (small), 32-36 mm (medium) and 38-46 mm (large), or for small valves (25-32 mm) and large valve (34 mm and above). Typically, the only difference between the different size prostheses is the length of the rigid posterior segment, which may be equal to the smallest number in the range of inter-trigone distances for a given size (such as 25 mm in the small size, and 34 mm or 38 mm, respectively, in the large sizes mentioned above. (All the division limits between small, medium, and large, or large and small are intuitive, and generally arbitrary.)

For some applications, as shown schematically in FIG. 15E, the surgeon measures a height D₂ of the anterior leaflet (A) of the mitral valve, as described hereinabove with reference to FIGS. 3A and 3C. Optionally, the surgeon also measures a height D₃ of the posterior leaflet (P) of the mitral valve, as described hereinabove with reference to FIGS. 3A and 3C. Responsively to the measured height D₂, and, optionally, the measured height D₃ (typically, the sum of D₂ and D₃), the surgeon ascertains a desired anterior-posterior height of prosthesis 600. For some applications, as described hereinabove with reference to FIGS. 6A-C, 7A-7C, and 8A-B and Equation 4, the surgeon ascertains the anterior-posterior height of prosthesis 600 responsively to the sum of D₂ and D₃) less a value indicative of twice a coaptation length, and typically minus the posterior bar curvature height H_{C}, as described hereinabove with reference to FIG. 3C, FIGS. 8A-B and Equation 5, and/or minus an additional constant to account for the depth of coaptation in the beating heart, as described hereinabove with reference to FIG. 8C and Equation 6 or Equation 7.

For some applications, prosthesis 600 is implanted as a complete annuloplasty prosthesis. For these applications, in order to achieve the desired anterior-posterior height of prosthesis 600, if necessary the surgeon trims first portion 652A of sleeve 650, as shown in FIG. 15F. The surgeon uses first anterior fiducial designators 664A to estimate the location at which the sleeve is to be trimmed. To do so, the surgeon uses the fiducial designators to measure a distance from location 672A on the sleeve (previously coupled to the annulus at the P1/P2 junction, as described above), which distance equals the desired anterior-posterior height of the prosthesis. Trimmed excess portion 676A of first portion 652A is discarded.

For example, assume the measured inter-trigone distance D₁ is 34 mm, and the desired anterior-posterior height of the prosthesis is calculated to be 40 mm Thus location 672A is half of inter-trigone distance D₁ from longitudinal center 636 of rigid posterior bar segment 630, i.e., 17 mm from the center. The location at which the sleeve is to be trimmed is thus the desired anterior-posterior height of the prosthesis (40 mm) from location 672A (which equals 57 mm from center 636).

After trimming the first portion, the surgeon couples the first portion of the sleeve at or near a trimmed anterior end thereof to the mitral valve at lateral fibrous trigone 110A, such as by suturing.

Similarly, the surgeon uses second anterior fiducial markers 664B to estimate the location at which the sleeve is to be coupled to medial fibrous trigone 110B. To do so, the surgeon uses the fiducial designators to measure a distance from location 672B on the sleeve (previously coupled to the annulus at the P2/P3 junction, as described above), which distance equals the desired anterior-posterior height of the prosthesis. The surgeon couples the second portion of the sleeve at this location to the mitral valve at medial fibrous trigone 110B, such as by suturing, as shown in FIG. 15F.

For example, assume the measured inter-trigone distance D₁ is 34 mm, and the desired anterior-posterior height of the prosthesis is calculated to be 40 mm. Thus location 672B is half of inter-trigone distance D₁ from longitudinal center 636 of rigid posterior bar segment 630, i.e., 17 mm from the center. The location at which the sleeve is to be coupled to medial fibrous trigone 110B is thus the desired anterior-posterior height of the prosthesis (40 mm) from location 672B (which equals 57 mm from center 636).

As shown in FIG. 15G, the surgeon then couples inter-trigone portion 668 between lateral and medial trigones 110A and 110B. The surgeon estimates the length of inter-trigone portion 668 to be equal to measured inter-trigone distance D₁. Fiducial designators 660 further include inter-trigone fiducial designators 669, anterior to second anterior fiducial markers 664B (inter-trigone fiducial designators 669 are typically located at least 80 mm beyond second bar end 632B). The surgeon uses inter-trigone fiducial designators 669 to measure the distance from the location at which the sleeve is coupled to medial fibrous trigone 110B. The surgeon trims any excess sleeve beyond the desired location. To continue the example above, the distance between the trimmed end of the sleeve (near medial fibrous trigone 110B) and the location at which the sleeve is to be coupled to lateral fibrous trigone 110A is 34 mm (which equals 91 mm from center 636). The surgeon then couples the sleeve at or near the trimmed end of the second portion, to the mitral valve at lateral trigone 110A, as shown in FIG. 15G. Optionally, the surgeon may elect to provide an overlapping portion between the ends of the sleeve, in which case the surgeon typically provides sufficient extra length to one or both of the sleeve ends when trimming the sleeve.

Alternatively, the sleeve is arranged such that first portion 652A is longer than second portion 652B, i.e., inter-trigone portion 668 begins at lateral trigone 110A and terminates at medial trigone 110B. This inverted arrangement may be less desirable for surgical reasons (the medial trigone is generally easier to see during surgery).

For other applications, prosthesis 600 is implanted as a band (i.e., partial ring). For these applications, in order to achieve the desired anterior-posterior height of prosthesis 600, in addition to trimming first portion 652A of sleeve 650, the surgeon also trims second portion 652B of sleeve 650, using second anterior fiducial designators 664B to estimate the proper location, as described hereinabove regarding trimming first portion 652A of sleeve 650. The surgeon couples the sleeve at or near the second trimmed end to medial fibrous trigone 110B, such as by suturing.

Based on the surgeon's surgical preferences, the surgeon may opt to suture the first and/or second portions 652A or 652B slightly posterior to trigones 110A or 110B, respectively (configuration not shown). In this case, the location on the sleeve is accordingly adjusted by reducing the calculated anterior-posterior height of prosthesis 600, to produce a shortened sleeve.

Although, for clarity of illustration, not shown in FIG. 15G, sleeve 650 is coupled (e.g., sutured) to the annulus of the mitral valve at additional locations along the sleeve, for example as shown in FIG. 2G, mutatis mutandis.

In a study performed by the inventor on porcine hearts, the inventor found that there was no significant change in valve symmetry during growth from piglets to adult pigs. The width of the P2 segment of the mitral valve annulus maintained a constant ratio to the inter-trigone distance, of about 0.9. 19 newborn piglets in the first two days of life and six adult pigs were studied. No animals were sacrificed for the purpose of this study; the piglets died accidently, and the adults were a sample of the regular product of a slaughter house. The animals were weighed, their hearts were carefully dissected under operative microscope using microsurgery tools, and weighed separately. The hearts were then suspended at the mitral valve plane. The valve constituents were identified and measured with a jeweler caliper. The measured parameters were the inter-trigone distance (ITD) and the width of the base of P2 (WP2). These measurements and ratios were compared to the same measurements and ratios in adult pigs, as shown in the following table:

TABLE 4

| | | Mean body | Mean heart | Mean WP2/ |
|---|---|---|---|---|
| | No. | mass (kg) | mass (g) | ITD (SD) |
| Piglets (newborn) | 19 | 1.133 | 7.93 | 0.85 (0.09) |
| Pigs | 6 | 320 | 425 | 0.93 (0.11) |
| Times growth | | 282 | 53.6 | 1.09 |
| P non-paired t-test | | | | NS |

The pigs were on average 282 times heavier than the piglets, and the pig hearts were on average 53.6 times heavier than piglet hearts. However, mitral valve symmetry, as measured by the ratio of the width of the base of P2 to the inter trigone distance (WP2/ITD) experienced only a minimal increase, which did not reach statistical significance.

Reference is now made to FIGS. 16A-B, which are schematic illustrations of a plication plasty technique for creating a fold in a posterior mitral valve leaflet, in accordance with an application of the present invention. In order to plan the segment of the posterior mitral leaflet to be reduced, and to achieve the proper height of the residual posterior leaflet 306, the surgeon draws an imaginary line 700 along posterior leaflet 306, parallel to its free margin, which leaves approximately 12-15 mm between the imaginary line and the free margin. All of posterior sutures 702 of the posterior part of an annuloplasty prosthesis 704, such as a partial or complete annuloplasty ring/band are pledgeted using everting mattress suturing. Every stitch is started from approximately 2-3 mm away from the leaflet-atrium line, on the atrial side in an atrium 706, as shown in FIG. 16A.

The needle points into the ventricle 300, and goes back up through posterior leaflet 306, passing through imaginary 700 line on posterior leaflet 306, thus marking the exact reduction of posterior leaflet 306. Sutures 702 are passed through annuloplasty prosthesis 704. Upon being tied, posterior pledgeted sutures 702 pull prosthesis 704 down in place, and plicate the part of the periphery of posterior leaflet 306 precisely as planned, leaving the spared part of the leaflet at the planned height. The sutures additionally provide excellent anchorage of the prosthesis to the posterior mitral annulus. FIG. 16B show a side view of an annuloplasty ring implanted using these techniques, while the heart is in systole.

For some applications, plication plasty is performed with needles oriented at right angles to the valve annulus (rather than along the rim, in other methods), which pass below the posterior leaflet to penetrate up along the middle of the leaflet (or as otherwise desired), and only then into the annuloplasty ring. Once tied down, the whole base of the posterior leaflet is plicated between the pledgets outside the rim and the annuloplasty ring more centrally, appearing as a long fold of leaflet beyond the annuloplasty ring. The entire plicated segment is gently but firmly hold by pledgets on the peripheral side, and the soft fabric of the annuloplasty ring (whereas in conventional cutting and sewing techniques, only simple sutures hold the cut edges). For some applications, this technique is practiced with a modified asymmetric profile of the suturing sleeve of the ring, such that the knots will not be in contact with the leaflet. (Alternatively, the knots are not directed away from the leaflet, and may at times contact the leaflet.) The pledgets, the ring, and the plicated leaflet sandwiched between them support each other, making the whole structure strong and suitably bulky. The ring device exerts centrally-oriented tension, which is best withstood by this bulky and pledgeted support, while reducing posterior leaflet height at the same time. It is not generally prone to dehiscence or breaks. It generally does not involve any leaflet resection. For some applications, the pledgets comprises Teflon felt. These techniques may be performed in combination with techniques described in Appendix A of U.S. Provisional Application 61/372,618, filed Aug. 11, 2011, which is incorporated herein by reference.

As used in the present application, including in the claims, an “annuloplasty prosthesis” is a prosthesis for supporting and stabilizing an annulus of an atrioventricular valve. The prosthesis may be a rigid, semi-rigid, and/or flexible annuloplasty ring or band, which may be complete or partial, unless specifically defined to have more particular characteristics.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-6. (canceled)

7. Apparatus comprising an annuloplasty prosthesis, which comprises:
a skeleton, which comprises a rigid posterior bar segment, which has first and second bar ends, and a length, measured in a straight line between the first and the second bar ends, of between 24 and 48 mm; and
a sleeve, which (a) comprises a flexible and substantially non-extensible fabric material, (b) covers the rigid posterior bar segment, (c) includes a first portion that extends at least 20 mm beyond the first bar end and a second portion that extends at least 25 mm beyond the second bar end, and (d) has a plurality of visually-sensible fiducial designators at respective longitudinal locations distributed along at least a portion of the sleeve, which fiducial designators are arranged to indicate a longitudinal center of the rigid posterior bar segment,
wherein the prosthesis is rigid along the rigid posterior bar segment, and is flexible or semi-rigid along at least 75% of the first portion of the sleeve and along at least 75% of the second portion of the sleeve.

8. The apparatus according to claim 7, wherein the fiducial designators include a central posterior fiducial designator located at the longitudinal center of the rigid posterior bar segment.

9-12. (canceled)

13. The apparatus according to claim 7, wherein the prosthesis is flexible or semi-rigid at all longitudinal locations other than along the rigid posterior bar segment.

14. The apparatus according to claim 7, wherein the prosthesis is flexible at all locations (a) beyond 15 mm beyond the first bar end and (b) beyond 15 mm beyond the second bar end.

15-19. (canceled)

20. The apparatus according to claim 7, wherein the skeleton further comprises first and second semi-rigid posterior segments, which begin at the first and the second bar ends, respectively, and extend away from a longitudinal center of the rigid posterior bar segment.

21-24. (canceled)

25. The apparatus according to claim 20, wherein the skeleton further comprises first and second flexible side segments, which begin at first and second anterior ends of the first and the second semi-rigid posterior segments, respectively, and extend away from the longitudinal center of the rigid posterior bar segment.

27. The apparatus according to claim 7, wherein a total length of the sleeve is between 100 and 180 mm.

28. The apparatus according to claim 7, wherein a length of the sleeve beyond the first bar end is between 20 and 50 mm, and a length of the sleeve beyond the second bar end is between 20 and 100 mm.

29-32. (canceled)

33. A method comprising:
measuring an inter-trigone distance between lateral and medial fibrous trigones of a native mitral valve of a subject; and
implanting an annuloplasty prosthesis at the native mitral valve such that the prosthesis restores a length of a middle scallop (P2) of a posterior leaflet of the mitral valve to the inter-trigone distance.

34-36. (canceled)

37. The method according to claim 33,
wherein the prosthesis includes (a) a skeleton, which comprises a rigid posterior bar segment, which has first and second bar ends, and a length of between 24 and 48 mm, and (b) a sleeve, which (i) comprises a flexible and substantially non-extensible fabric material, (ii) covers the rigid posterior bar segment, and (iii) includes a first portion that extends at least 20 mm beyond the first bar end and a second portion that extends at least 20 mm beyond the second bar end, and
wherein implanting comprises coupling the prosthesis, at a plurality of locations along the sleeve, to a posterior leaflet annulus of the native mitral valve.

38. (canceled)

39. The method according to claim 37, wherein coupling the sleeve comprises:
selecting two of the locations along the sleeve that are on opposite longitudinal sides of a longitudinal center of the rigid posterior bar segment; and
coupling the sleeve at the two locations to two sides of the middle scallop (P2) of the posterior leaflet annulus directly opposite small commissures at:
a junction of a lateral scallop (P1) of the posterior leaflet and P2, and
a junction of P2 and a medial scallop (P3) of the posterior leaflet, respectively.

40. The method according to claim 39, wherein coupling further comprises, prior to coupling the sleeve at the two locations, coupling the sleeve at the longitudinal center of the rigid posterior bar segment to a middle of the P2 annulus.

41-46. (canceled)

47. The method according to claim 37, further comprising:
measuring a height of an anterior leaflet of the native mitral valve; and
responsively to the measured height, trimming the first and the second portions of the sleeve if necessary.

48. The method according to claim 47, further comprising measuring a height of a posterior leaflet of the native mitral valve, wherein trimming comprises trimming responsively to both the measured height of the anterior leaflet and the measured height of the posterior leaflet.

49-61. (canceled)

62. A method comprising:
providing an annuloplasty prosthesis that includes (a) a skeleton, which comprises a rigid posterior bar segment, which has first and second bar ends, and a length, measured in a straight line between the first and the second bar ends, of between 24 and 48 mm, and (b) a sleeve, which (i) comprises a flexible and substantially non-extensible fabric material, (ii) covers the rigid posterior bar segment, and (iii) includes a first portion that extends at least 20 mm beyond the first bar end and a second portion that extends at least 20 mm beyond the second bar end;
measuring an inter-trigone distance between lateral and medial fibrous trigones of a native mitral valve of a subject;
responsively to the measured inter-trigone distance, selecting two locations along the sleeve that are on opposite longitudinal sides of a longitudinal center of the rigid posterior bar segment; and
implanting the annuloplasty prosthesis at the native mitral valve by coupling the prosthesis to a posterior leaflet of the native mitral valve, including at the two locations along the sleeve.

63-64. (canceled)

65. The method according to claim 62, wherein coupling the sleeve comprises coupling the sleeve at the two of the locations along the sleeve to two sides of a middle scallop (P2) of the posterior leaflet directly opposite small commissures at:
a junction of a lateral scallop (P1) of the posterior leaflet and P2, and
a junction of P2 and a medial scallop (P3) of the posterior leaflet, respectively.

66. The method according to claim 62, wherein coupling further comprises, prior to coupling the sleeve at the two locations, coupling the sleeve at a longitudinal center of the rigid posterior bar segment to a middle of a middle scallop (P2) annulus.

67. (canceled)

68. The method according to claim 62,
wherein the sleeve has a plurality of visually-sensible fiducial designators at respective longitudinal locations distributed along at least a portion of the sleeve, and
wherein selecting the two locations along the sleeve comprises selecting the two locations along the sleeve using at least two of the fiducial designators.

69-78. (canceled)

79. The method according to claim 62, wherein the prosthesis is flexible or semi-rigid at all longitudinal locations other than along the rigid posterior bar segment.

80-81. (canceled)

82. The method according to claim 62, wherein the skeleton further includes first and second semi-rigid posterior segments, which begin at the first and the second bar ends, respectively, and extend away from a longitudinal center of the rigid posterior bar segment.

83-160. (canceled)
